(19) United States
(12) Patent Application Publication
(10) Pub. No.: US 2026/0136830 A1
(43) Pub. Date: May 14, 2026

(54) COMPOUND, ORGANIC THIN FILM, PHOTOELECTRIC CONVERSION ELEMENT, IMAGE SENSOR, PHOTOSENSOR AND SOLID-STATE IMAGING APPARATUS

(71) Applicant: MITSUBISHI GAS CHEMICAL COMPANY, INC., Tokyo (JP)

(72) Inventors: Toshinari AOKI, Tokyo (JP); Tatsuro TAKAMURA, Tokyo (JP); Masahiro SHIMIZU, Tokyo (JP); Takashi YAMAMOTO, Tokyo (JP)

(73) Assignee: MITSUBISHI GAS CHEMICAL COMPANY, INC., Tokyo (JP)

(21) Appl. No.: 19/145,003

(22) PCT Filed: Apr. 8, 2024
(86) PCT No.: PCT/JP2024/014314

§ 371 Date: Jan. 22, 2026

(30) Foreign Application Priority Data
Apr. 20, 2023 (JP) 2023-069028

(51) Int. Cl. H10K 85/60; C07D 471/04; H10K 39/32

(52) U.S. Cl. CPC H10K 85/6572; C07D 471/04; H10K 39/32; H10K 85/6574

(57) ABSTRACT

Provided is a compound represented by the following formula (1):

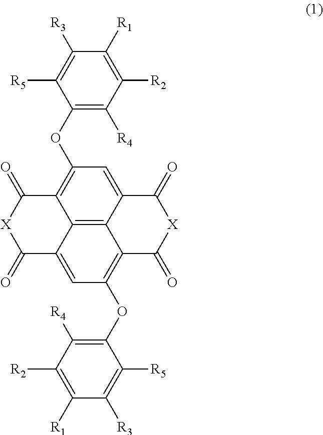

wherein X is an oxygen atom or NR₆, and R₁ to R₆ are each independently a hydrogen atom, a halogen atom, a cyano group, a linear, branched or cyclic alkyl group, an aryl group, or the like.

### TECHNICAL FIELD

The present disclosure relates to a compound, an organic thin film, a photoelectric conversion element, an image sensor, a photosensor and a solid-state imaging apparatus.

### BACKGROUND ART

Techniques of converting visible light to electric signals by photoelectric conversion have heretofore been known and are used in, for example, image sensors. Such image sensors are carried by solid-state imaging apparatuses such as CCD (charge coupled device) image sensors and CMOS (complementary metal oxide semiconductor) image sensors. In recent years, reduction in pixel size have been underway in solid-state imaging apparatuses, and organic photoelectric conversion films adapted therefor have been studied. For example, Patent Documents 1 and 2 each disclose an organic photoelectric conversion film constituted by subphthalocyanine and an imide.

### CITATION LIST

### Patent Document

Patent Document 1: Japanese Patent Application Laid-Open No. 2018-32754
Patent Document 2: Japanese Translation of PCT International Application Publication No. 2018-512423
Patent Document 3: Japanese Translation of PCT International Application Publication No. 2014-506736

### SUMMARY OF INVENTION

### Technical Problem

Solid-state imaging apparatuses are required to achieve both high spectral selectivity and a high S/N ratio. Hence, the solid-state imaging apparatuses are desired to have high external quantum efficiency (EQE) and low dark current characteristics. An approach of disposing an electron transport layer and a hole blocking layer, and/or a hole transport layer and an electron blocking layer between a photoelectric conversion unit and an electrode unit is known in order to achieve both of such factors. In this context, each of the electron transport layer, the hole blocking layer and the electron blocking layer, etc. widely used in the field of organic electronic devices is disposed at the interface between an electrode or a film having conductivity and other films in films constituting a device. These layers play a role in controlling the back transfer of holes or electrons and adjusting the leakage of unnecessary holes or electrons. For example, Patent Document 3 discloses an example using 1,4,5,8-naphthalene-tetracarboxylic dianhydride (NTCDA) as a material for use in such a layer.

However, conventional hole blocking layers and electron blocking layers including those disclosed in Patent Document 3 are still susceptible to improvement in the suppression of leak current in the dark.

An object of the present invention is to provide a novel compound that can suppress leak current in the dark and is useful, particularly, in a photoelectric conversion element material, and a photoelectric conversion element material as well as an organic thin film, a photoelectric conversion element, an image sensor, a photosensor and a solid-state imaging apparatus including the compound.

### Solution to Problem

The present invention is as described below.

[1]

A compound represented by the following formula (1):

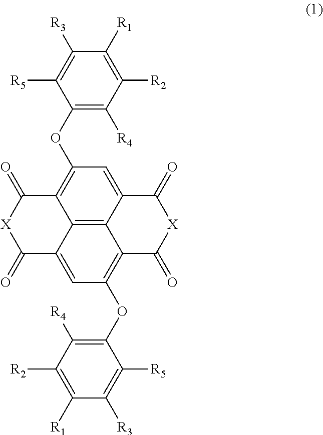

wherein
X is an oxygen atom or NR₆,
R₁, R₂, R₃, R₄, R₅ and R₆ are each independently selected from the group consisting of a hydrogen atom, a halogen atom, a hydroxy group, a thiol group, an amino group, a cyano group, a carboxy group, a nitro group, and an optionally substituted linear, branched or cyclic alkyl group, thioalkyl group, thioaryl group, arylsulfonyl group, aryloxy group, alkylsulfonyl group, alkylamino group, arylamino group, alkoxy group, acylamino group, acyloxy group, aryl group, carboxyamide group, carboalkoxy group, carboaryloxy group, acyl group, and monovalent heterocyclic group, any adjacent members among R₁, R₂, R₃, R₄, R₅ and R₆ optionally constitute a portion of a condensed aliphatic ring or a condensed aromatic ring, and two R₁, R₂, R₃, R₄, R₅ or X moieties are the same as or different from each other, wherein the condensed aliphatic ring and the condensed aromatic ring each optionally contain one or more atoms other than carbon.
   [2]

The compound according to [1], wherein an energy level of the lowest unoccupied molecular orbital obtained by density functional formalism of the compound represented by the formula (1) is −6.00 eV or more and −3.40 eV or less.

[3]

The compound according to [1], wherein a difference between an energy level of the lowest unoccupied molecular orbital and an energy level of the highest occupied molecular orbital obtained by density functional formalism of the compound represented by the formula (1) is 3.00 eV or more and 4.00 eV or less.

[4]

The compound according to [1], wherein the compound is a material for a photoelectric conversion element.

[5]

An organic thin film comprising the compound according to [1].

[6]

The organic thin film according to [5], wherein the organic thin film has a local maximum absorption wavelength of an optical absorption band at 450 nm or less.

[7]

A photoelectric conversion element comprising a first electrode film, a second electrode film, and a photoelectric conversion film positioned between the first electrode film and the second electrode film, wherein
the photoelectric conversion film comprises the material for a photoelectric conversion element according to [4].
   [8]

A photoelectric conversion element comprising a first electrode film, a second electrode film, and a photoelectric conversion film positioned between the first electrode film and the second electrode film, wherein
the photoelectric conversion film comprises the organic thin film according to [5].
   [9]

The photoelectric conversion element according to [7] or [8], wherein
the photoelectric conversion film comprises a photoelectric conversion layer and an auxiliary layer, wherein
the auxiliary layer is made of only the organic thin film or made of a plurality of films including the organic thin film.
   [10]

An image sensor comprising the photoelectric conversion element according to [7] or [8].

[11]

The image sensor according to [10], wherein the image sensor is prepared by laminating two or more photoelectric conversion elements.

[12]

An image sensor prepared by disposing a plurality of photoelectric conversion elements according to [7] or [8] in an array pattern.

[13]

A photosensor comprising the image sensor according to [10].

[14]

A solid-state imaging apparatus comprising the image sensor according to [10].

### Advantageous Effects of Invention

The present invention can provide a novel compound useful, particularly, in a photoelectric conversion element material, a photoelectric conversion element material, and an organic thin film, a photoelectric conversion element, an image sensor, a photosensor and a solid-state imaging apparatus comprising the compound.

### BRIEF DESCRIPTION OF DRAWINGS

FIG. 1 is a cross-sectional schematic view partially showing one example of the photoelectric conversion element of the present invention.

### DESCRIPTION OF EMBODIMENTS

Hereinafter, the mode for carrying out the present invention (hereinafter, simply referred to as the “present embodiment”) will be described in detail with reference to the drawings, if necessary. However, the present invention is not limited by the present embodiment described below. Various changes or modifications can be made in the present invention without departing from the spirit of the present invention. In the drawings, the same numerals or symbols will be used to designate the same components, so that the description will be omitted. Positional relationship indicated by terms such as “up”, “down”, “right” and “left” is based on the positional relationship shown in the drawings, unless otherwise specified. The dimensional ratios of the drawings are not limited to the shown ratios.

### (Compound)

The compound of the present embodiment is represented by the following formula (1) (hereinafter, this compound is also referred to as a “compound (1)”):

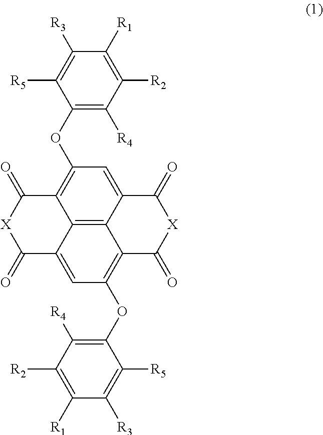

In the formula, X is an oxygen atom or NR₆, R₁, R₂, R₃, R₄, R₅ and R₆ are each independently selected from the group consisting of a hydrogen atom, a halogen atom, a hydroxy group, a thiol group, an amino group, a cyano group, a carboxy group, a nitro group, and an optionally substituted linear, branched or cyclic alkyl group, thioalkyl group, thioaryl group, arylsulfonyl group, aryloxy group, alkylsulfonyl group, alkylamino group, arylamino group, alkoxy group, acylamino group, acyloxy group, aryl group, carboxyamide group, carboalkoxy group, carboaryloxy group, acyl group, and monovalent heterocyclic group, and any adjacent members among R₁, R₂, R₃, R₄, R₅ and R₆ optionally constitute a portion of a condensed aliphatic ring or a condensed aromatic ring. Two R₁, R₂, R₃, R₄, R₅ or X moieties are the same as or different from each other. The condensed aliphatic ring and the condensed aromatic ring each optionally contain one or more atoms other than carbon.

Such a compound (1) can suppress leak current in the dark and exhibits excellent characteristics, particularly, as a photoelectric conversion element material. A factor for this is not certain and is considered as follows by the present inventors, though the factor is not limited thereto: the compound (1) has two aryloxy groups in a molecular structure, and these two aryloxy groups are diagonally positioned with respect to a naphthyl group, whereby the energy level of the lowest unoccupied molecular orbital of the compound (1) is decreased, and leak current in the dark can be suppressed. Also, the compound (1) has two aryloxy groups, whereby the compound (1) has low crystallinity, easily has a plurality of stable structures having similar energy levels, and is therefore unlikely to assume molecular arrangement having high regularity as a solid. This can suppress the generation of a disturbed film thickness and an electric short circuit (pinhole) that may occur due to partial aggregation in a thin film. Furthermore, the compound (1), as compared with the absence of two aryloxy groups, has low molecular orientation in the layer direction in a thin film, and therefore produces high electron-transporting properties and produces excellent characteristics as a photoelectric conversion element material.

Examples of the halogen atom include a fluorine atom (F), a chlorine atom (Cl), a bromine atom (Br) and an iodine atom (I).

The linear alkyl group may be a linear alkyl group in which the number of carbon atoms in the alkyl group is 1 to 12. Examples thereof include a methyl group (Me), an ethyl group (Et), a n-propyl group (n-Pr), a n-butyl group (n-Bu), a n-pentyl group, a n-hexyl group, a n-heptyl group, a n-octyl group, a n-nonyl group, a n-decyl group and n-dodecyl group. Among them, the linear alkyl group is preferably a linear alkyl group having 1 to 3 carbon atoms, more preferably a methyl group or an ethyl group, further preferably a methyl group, from the viewpoint of more effectively and reliably exerting the working effects according to the present invention.

The branched alkyl group may be a branched alkyl group in which the number of carbon atoms in the alkyl group is 1 to 12. Examples thereof include an isopropyl group (i-Pr), a sec-butyl group (s-Bu), a tert-butyl group (t-Bu), an isopentyl group, a sec-pentyl group, a 3-pentyl group, a neopentyl group, an isohexyl group, an isooctyl group, an isononyl group, an isodecyl group and isododecyl group. The linear or branched alkyl group may have a substituent. Examples of the substituent include halogen atoms such as a fluorine atom, monovalent groups having an aromatic ring such as a benzyl group, a naphthyl group and a phenoxy group, monovalent groups having a heteroatom such as alkoxy groups, aminoalkyl and thioalkyl groups, monovalent groups having a heterocyclic ring such as a pyridyl group, a hydroxy group, a carboxyl group, an amino group, and a thiol group.

The cyclic alkyl group may be a cyclic alkyl group in which the number of carbon atoms in the alkyl group is 3 to 10. Examples thereof include a cyclopropyl group, a cyclobutyl group, a cyclopentyl group, a cyclohexyl group, a cycloheptyl group and a cyclooctyl group. The cyclic alkyl group may have a heteroatom such as a nitrogen atom, an oxygen atom, and/or a sulfur atom in its ring. Examples of such a cyclic alkyl group include a pyrrolidinyl group, an oxazolidinyl group, a pyrazolidinyl group, a thiazolidinyl group, an imidazolidinyl group, a dioxofuranyl group, a tetrahydrofuranyl group, a tetrahydrothiophenyl group, a piperazinyl group, a dioxanyl group, and a morpholinyl group. A monovalent group such as a hydroxy group, a carboxyl group, an amino group and/or a thiol group may be further bonded to the cyclic alkyl group.

The thioalkyl group (—SR; hereinafter, R represents an alkyl group) and the thioaryl group (—SAr; hereinafter, Ar represents an aryl group) may be a thioalkyl group in which the number of carbon atoms in the alkyl group is 1 to 12 and a thioaryl group in which the number of carbon atoms in the aryl group is 6 to 16. The thioalkyl group and the thioaryl group may further have a substituent including an amino group, a hydroxy group, halogen atoms, alkoxy groups, and thioalkyl groups. Examples of such a thioalkyl group and a thioaryl group include a methylthio group, an ethylthio group, a phenylthio group, a toluylthio group, an aminophenylthio group, a hydroxyphenylthio group, a fluorophenylthio group, a dimethylphenylthio group, and a methylthiophenylthio group.

The arylsulfonyl group (—SO₂—Ar) may be an arylsulfonyl group in which the number of carbon atoms in the aryl group is 6 to 16. Examples thereof include a phenylsulfonyl group, a toluenesulfonyl group, a dimethylbenzenesulfonyl group, a mesitylenesulfonyl group, an octylbenzenesulfonyl group, and a naphthalenesulfonyl group.

The aryloxy group (—O—Ar) may be an aryloxy group in which the number of carbon atoms in the aryl group is 6 to 16. The aryloxy group may further have a substituent including a cyano group, halogen atoms such as a fluorine atom, a hydroxy group, alkoxy groups such as a methoxy group, an amino group, alkylamino groups, a thiol group, and aryloxy groups. Examples of such an aryloxy group include a phenoxy group, a cyanophenoxy group, a methylcyanophenoxy group, a dimethylcyanophenoxy group, a fluorocyanophenoxy group, a dicyanophenoxy group, a methoxycyanophenoxy group, a tricyanophenoxy group, a cyanonaphthoxy group, a dicyanonaphthoxy group, a 2-methylphenoxy group, a 3-methylphenoxy group, a 4-methylphenoxy group, a fluoromethylphenoxy group, a dimethylphenoxy group, a 3-hydroxyphenoxy group, a fluoro-3-hydroxyphenoxy group, a 2-hydroxyphenoxy group, a fluoro-2-hydroxyphenoxy group, a methoxyphenoxy group, an ethoxyphenoxy group, a fluorophenoxy group, a perfluorophenoxy group, a dimethoxyphenoxy group, an aminophenoxy group, a N,N-dimethylaminophenoxy group, a thiophenoxy group, a (trifluoromethyl)phenoxy group, a naphthoxy group, a methoxynaphthoxy group, a fluoronaphthoxy group, and a phenoxyphenoxy group.

The alkylsulfonyl group (—SO₂—R) may be an alkylsulfonyl group in which the number of carbon atoms in the alkyl group is 1 to 12. Examples thereof include a mesyl group, an ethylsulfonyl group, and a n-butylsulfonyl group.

The alkylamino group (wherein the alkylamino group is —NHR or —NR₂ wherein two R moieties may be the same as or different from each other) may be an alkylamino group in which the number of carbon atoms in the alkyl group is 1 to 12. Examples thereof include a methylamino group, an ethylamino group, a n-propylamino group, a n-butylamino group, a n-pentylamino group, a n-hexylamino group, a n-heptylamino group, a n-octylamino group, a n-nonylamino group, a n-decylamino group, a n-dodecylamino group, an isopropylamino group, a sec-butylamino group, a tert-butylamino group, an isopentylamino group, a sec-pentylamino group, a 3-pentylamino group, a neopentylamino group, an isohexylamino group, an isoheptylamino group, an isooctylamino group, an isononylamino group, an isodecylamino group and isododecylamino group, a dimethylamino group, a diethylamino group, a diisopropylamino group, and an isopropylethylamino group.

The arylamino group (wherein the arylamino group is —NHAr or —NAr₂ wherein two Ar moieties may be the same as or different from each other) may be an arylamino group in which the number of carbon atoms in the aryl group is 6 to 16. Examples thereof include an anilino group, a toluidinyl group, a dimethylanilino group, an isopropylanilino group, a t-butylanilino group, a fluoroanilino group, a trifluoromethylanilino group, a bis(trifluoromethyl)anilino group, a pyridylamino group, a methylpyridylamino group, a fluoropyridylamino group, a pyrimidylamino group, and a biphenylamino group.

The alkoxy group (—OR) may be an alkoxy group having 1 to 12 carbon atoms. Examples thereof include a methoxy group, an ethoxy group, a n-propoxy group, a n-butyloxy group, a n-pentoxy group, a n-hexoxy group, a n-heptoxy group, a n-octoxy group, a n-nonoxy group, a n-decoxy group and n-dodecoxy group, an isopropoxy group, a sec-butyloxy group, a tert-butyloxy group, an isopentoxy group, a sec-pentoxy group, a 3-pentoxy group, a neopentoxy group, an isohexoxy group, an isooctoxy group, an isononoxy group, an isodecoxy group, and an isododecoxy group.

The acylamino group (—NH—COR or —NH—COAr) may be a group in which the number of carbon atoms in the alkyl group is 1 to 12 or the number of carbon atoms in the aryl group is 6 to 16, and may have a substituent including halogen atoms such as a fluorine atom, alkoxy groups, and a cyano group. Examples of such an acylamino group include an acetylamino group, a propionylamino group, a benzoylamino group, a methylbenzoylamino group, a dimethylbenzoylamino group, a methoxybenzoylamino group, a cyanobenzoylamino group, and a bis(trifluoromethyl)benzoylamino group.

The acyloxy group (—O—COR or —O—COAr) may be a group in which the number of carbon atoms in the alkyl group is 1 to 12 or the number of carbon atoms in the aryl group is 6 to 16. The acyloxy group may further have a substituent including halogen atoms such as a fluorine atom, and a cyano group and may have a heteroatom such as a nitrogen atom in an aromatic ring. Examples of such an acyloxy group include a benzoyloxy group, a toluoyloxy group, a dimethylbenzoyloxy group, a cyanobenzoyloxy group, a fluorobenzoyloxy group, a bis(trifluoromethyl)benzoyloxy group, a pyridinecarboxy group, and a methylpyridinecarboxy group.

The aryl group (—Ar) may be an aryl group having 6 to 16 carbon atoms. The aryl group may further have a substituent including an amino group, a hydroxy group, a thiol group, halogen atoms such as a fluorine atom, a nitro group, and a cyano group and may have a heteroatom such as a nitrogen atom in an aromatic ring. Examples of such an aryl group include a phenyl group, a methylphenyl group, an ethylphenyl group, a dimethylphenyl group, a trimethylphenyl group, a methoxyphenyl group, a dimethoxyphenyl group, a trimethoxyphenyl group, a methoxymethylphenyl group, an aminophenyl group, a diaminophenyl group, an aminomethylphenyl group, a hydroxyphenyl group, a dihydroxyphenyl group, a hydroxymethylphenyl group, a hydroxyethylphenyl group, a thiophenyl group, a methylthiophenyl group, a dithiophenyl group, a fluorophenyl group, a fluoromethylphenyl group, a trifluoromethylphenyl group, a perfluorophenyl group, a fluoro(trifluoromethyl)phenyl group, a bis(trifluoromethyl)phenyl group, a cyanophenyl group, a methylcyanophenyl group, a dimethylcyanophenyl group, a dicyanophenyl group, a methoxycyanophenyl group, a tricyanophenyl group, a dicyanophenyl group, a methylcyanopyridyl group, a (trifluoromethyl)cyanopyridyl group, a dimethylcyanopyridyl group, a dicyanopyridyl group, a methoxycyanopyridyl group, a tricyanopyridyl group, a cyanopyridyl group, a naphthyl group, a nitrophenyl group, a dinitrophenyl group, a nitrofluorophenyl group, a methylnaphthyl group, an ethylnaphthyl group, a dimethylnaphthyl group, a trimethylnaphthyl group, a methoxynaphthyl group, a dimethoxynaphthyl group, a trimethoxynaphthyl group, an aminonaphthyl group, a diaminonaphthyl group, an aminomethylnaphthyl group, a hydroxynaphthyl group, a dihydroxynaphthyl group, a hydroxymethylnaphthyl group, a hydroxyethylnaphthyl group, a thionaphthyl group, a methylthionaphthyl group, a dithionaphthyl group, a fluoronaphthyl group, a trifluoromethylnaphthyl group, a perfluoronaphthyl group, a di(trifluoromethyl)naphthyl group, a biphenyl group, and a cyanobiphenyl group.

The carboxyamide group (wherein the carboxyamide group is —CO—NH₂, —CO—NHR, —CONR₂ wherein two R moieties may be the same as or different from each other, —CONHAr, or —CONAr₂ wherein two Ar may be the same as or different from each other) may be a carboxyamide group in which the number of carbon atoms in the alkyl group is 1 to 12 or the number of carbon atoms in the aryl group is 6 to 16. Examples thereof include a dimethylcarboxyamide group and a diphenylcarboxyamide group.

The carboalkoxy group or the carboaryloxy group (—COOR or —COOAr) may be a carboalkoxy group in which the number of carbon atoms in the alkyl group is 1 to 12 or the number of carbon atoms in the aryl group is 6 to 16. Examples thereof include a carbomethoxy group and a carbophenoxy group.

The monovalent heterocyclic group may be a monovalent heterocyclic group having 3 to 14 carbon atoms. Examples thereof include a furanyl group, a thienyl group, a pyrrolyl group, a pyrazolyl group, an imidazolyl group, a triazolyl group, an oxazolyl group, a dioxazolyl group, an isoxazolyl group, an oxadiazolyl group, a thiazolyl group, an isothiazolyl group, a thiadiazolyl group, a triazolyl group, an indolyl group, an indolinyl group, an indolizinyl group, an indazolinyl group, indoleninyl group, a benzofuranyl group, a benzothienyl group, a carbazolyl group, a dibenzofuranyl group, a dibenzothienyl group, a pyridinyl group, a diazinyl group, an oxazinyl group, a thiazinyl group, a dioxinyl group, a dithienyl group, a triazinyl group, a pyrimidinyl group, a pyrazinyl group, a pyridazinyl group, a quinolinyl group, an isoquinolinyl group, a cinnolinyl group, a phthalazinyl group, a quinazolinyl group, a naphthyridinyl group, a purinyl group, a pteridinyl group, an acridinyl group, a phenanthridinyl group, a phenanthrolinyl group, a xanthenyl group, a phenoxazinyl group, a thianthrenyl group, a morpholinyl group and a phenazinyl group.

In the compound (1) of the present embodiment, without particular limitations, R₁, R₂, R₃, R₄ and R₅ are preferably each independently selected from the group consisting of a hydrogen atom, a halogen atom, a hydroxy group, a thiol group, an amino group, a cyano group, a carboxy group, a nitro group, and an optionally substituted linear, branched or cyclic alkyl group, thioalkyl group, thioaryl group, arylsulfonyl group, aryloxy group, alkylsulfonyl group, alkylamino group, arylamino group, alkoxy group, acylamino group, acyloxy group, aryl group, carboxyamide group, carboalkoxy group, carboaryloxy group, acyl group, and monovalent heterocyclic group, more preferably each independently selected from the group consisting of a hydrogen atom, a halogen atom, a nitro group, a cyano group, and an optionally substituted linear, branched or cyclic alkyl group, further preferably each independently selected from the group consisting of a hydrogen atom, a halogen atom, a nitro group, a cyano group, an unsubstituted alkyl group, and a halogen atom-substituted alkyl group, particularly preferably each independently selected from the group consisting of a cyano group and an unsubstituted alkyl group, and exceedingly preferably a cyano group. The compound (1) having the structure mentioned above can suppress leak current in the dark.

In the present embodiment, R₁, R₂, R₃, R₄ and R₅ may be the same or different. Without particular limitations, at least two moieties selected from R₁, R₂, R₃, R₄ and R₅ are preferably the same, and at least four moieties selected from R₁, R₂, R₃, R₄ and R₅ are more preferably the same, from the viewpoint of more effectively and reliably exerting the working effects according to the present invention. Two R₁, R₂, R₃, R₄, or R₅ moieties in the compound (1) may be the same as or different from each other and are preferably the same as each other.

In the compound (1) of the present embodiment, without particular limitations, at least one of R₁, R₂, R₃, R₄ and R₅ is preferably not a hydrogen atom, and at least one of R₁, R₂, R₃, R₄ and R₅ is more preferably selected from the group consisting of a halogen atom, a nitro group, a cyano group, and an optionally substituted linear, branched or cyclic alkyl group, from the viewpoint of more effectively and reliably exerting the working effects according to the present invention. At least one of R₁, R₂, R₃, R₄ and R₅ is further preferably selected from the group consisting of a halogen atom, a nitro group, a cyano group, an unsubstituted alkyl group, and a halogen atom-substituted alkyl group, particularly preferably selected from the group consisting of a cyano group and an unsubstituted alkyl group, and exceedingly preferably a cyano group.

In the compound (1) of the present embodiment, without particular limitations, at least two of R₁, R₂, R₃, R₄ and R₅ are preferably hydrogen atoms, at least two or at least three of R₁, R₂, R₃, R₄ and R₅ are more preferably hydrogen atoms, and at least four of R₁, R₂, R₃, R₄ and R₅ are particularly preferably hydrogen atoms, from the viewpoint of more effectively and reliably exerting the working effects according to the present invention.

In the present embodiment, when at least four of R₁, R₂, R₃, R₄ and R₅ are hydrogen atoms, without particular limitations, each of R₂, R₃, R₄ and R₅ is preferably a hydrogen atom from the viewpoint of more effectively and reliably exerting the working effects according to the present invention.

When each of R₂, R₃, R₄ and R₅ is a hydrogen atom, R₁ is preferably selected from the group consisting of a halogen atom, a hydroxy group, a thiol group, an amino group, a cyano group, a carboxy group, a nitro group, and an optionally substituted linear, branched or cyclic alkyl group, thioalkyl group, thioaryl group, arylsulfonyl group, aryloxy group, alkylsulfonyl group, alkylamino group, arylamino group, alkoxy group, acylamino group, acyloxy group, aryl group, carboxyamide group, carboalkoxy group, carboaryloxy group, acyl group, and monovalent heterocyclic group, more preferably selected from the group consisting of a halogen atom, a nitro group, a cyano group, and an optionally substituted linear, branched or cyclic alkyl group, further preferably selected from the group consisting of a halogen atom, a nitro group, a cyano group, an unsubstituted alkyl group, and a halogen atom-substituted alkyl group, particularly preferably selected from the group consisting of a cyano group and an unsubstituted alkyl group, and exceedingly preferably a cyano group.

In the compound (1) of the present embodiment, X is an oxygen atom or NR₆, and two X moieties may be the same as or different from each other and are preferably the same as each other from the viewpoint of more effectively and reliably exerting the working effects according to the present invention. When X is NR₆, without particular limitations, R₆ is preferably selected from the group consisting of a hydrogen atom, a halogen atom, a hydroxy group, a thiol group, an amino group, a cyano group, a carboxy group, a nitro group, and an optionally substituted linear, branched or cyclic alkyl group, thioalkyl group, thioaryl group, arylsulfonyl group, aryloxy group, alkylsulfonyl group, alkylamino group, arylamino group, alkoxy group, acylamino group, acyloxy group, aryl group, carboxyamide group, carboalkoxy group, carboaryloxy group, acyl group, and monovalent heterocyclic group, more preferably selected from the group consisting of an optionally substituted linear, branched or cyclic alkyl group, aryl group, and monovalent heterocyclic group, particularly preferably an aryl group optionally substituted by a group selected from the group consisting of a halogen atom, a nitro group, a cyano group, and a halogen atom-substituted alkyl group. The compound (1) having the structure mentioned above can more suppress leak current in the dark. When both of two X moieties in the compound (1) are NR₆, two R₆ moieties may be the same as or different from each other and are preferably the same as each other from the viewpoint of more effectively and reliably exerting the working effects according to the present invention.

The energy level of the lowest unoccupied molecular orbital (LUMO) obtained by density functional formalism of the compound (1) of the present embodiment is preferably −6.00 eV or more and −3.40 eV or less, more preferably −5.50 eV or more and −3.45 eV or less, from the viewpoint of more effectively and reliably exerting the working effects according to the present invention. This energy level may be −6.00 eV or more and −3.80 eV or less or may be −5.50 eV or more and −3.80 eV or less. The compound (1) of the present embodiment can be structurally optimized by molecular simulation using density functional formalism (e.g., molecular simulation using quantum chemical calculation program Gaussian manufactured by Gaussian, Inc.) to determine the energy level of the lowest unoccupied molecular orbital of the compound (1). The energy level of the lowest unoccupied molecular orbital obtained by density functional formalism of the compound (1) of the present embodiment may be adjusted by changing R₁ to R₆. At least one of R₁, R₂, R₃, R₄ and R₅ is preferably an electron-withdrawing group, and R₆ is preferably an electron-withdrawing group-substituted aryl group, from the viewpoint of setting the energy level of the lowest unoccupied molecular orbital to within the range described above.

The energy level of the highest occupied molecular orbital (HOMO) obtained by density functional formalism of the compound (1) of the present embodiment is preferably −8.00 eV or more and −6.00 eV or less, more preferably −7.90 eV or more and −6.10 eV or less, from the viewpoint of more effectively and reliably exerting the working effects according to the present invention. The compound (1) of the present embodiment can be structurally optimized by molecular simulation using density functional formalism (e.g., molecular simulation using quantum chemical calculation program Gaussian manufactured by Gaussian, Inc.) to determine the energy level of the highest occupied molecular orbital. The energy level of the highest occupied molecular orbital obtained by density functional formalism of the compound (1) of the present embodiment may be adjusted, without particular limitations, by changing R₁ to R₆. Without particular limitations, at least one of R₁, R₂, R₃, R₄ and R₅ is preferably an electron-withdrawing group, and R₆ is preferably an electron-withdrawing group-substituted aryl group, from the viewpoint of setting the energy level of the highest occupied molecular orbital to within the range described above.

The difference (eV) between the energy level of the lowest unoccupied molecular orbital and the energy level of the highest occupied molecular orbital ([energy level of the highest occupied molecular orbital]−[energy level of the lowest unoccupied molecular orbital]) obtained by density functional formalism of the compound (1) of the present embodiment is preferably 3.00 eV or more and 4.00 eV or less. The compound (1) having the difference in energy level that falls within the range described above tends to be able to reduce leak current in the dark when used as a photoelectric conversion element material.

The molecular weight of the compound (1) of the present embodiment is preferably 400 or higher, more preferably 430 or higher, further preferably 450 or higher. When the molecular weight is 400 or higher, change in physical properties caused by the thermal motion of molecules that may occur in heating operation in an organic thin film production process or a high-temperature environment using the compound (1) can be more suppressed. Particularly, in the case of forming the compound (1) by vacuum vapor deposition, the molecular weight of the compound (1) is preferably 1000 or lower, more preferably 950 or lower, further preferably 900 or lower. When the molecular weight is 1000 or lower, heat energy necessary for sublimation in forming an organic thin film of the compound (1) by vacuum vapor deposition can be kept lower. A favorable thin film can thereby be formed without thermally deteriorating the compound (1). However, in the case of forming a thin film by solution coating, the molecular weight of the compound (1) may be larger than 1000 because such a problem is unlikely to occur.

In the compound (1) of the present embodiment, the temperature at which the weight ratio of weight reduction ascribable to heating in an inert gas atmosphere falls within 5% of the weight before heating (hereinafter, also referred to as the “5% weight reduction temperature”) is preferably 200° C. or higher, more preferably 250° C. or higher. When the 5% weight reduction temperature is 200° C. or higher, change in physical properties caused by the thermal motion of molecules that may occur in heating operation in an organic thin film production process or a high-temperature environment using the compound (1) can be more suppressed. The 5% weight reduction temperature can be measured by differential thermal analysis.

The compound (1) of the present embodiment is obtained by synthesis, for example, as mentioned later. The content ratio of the compound (1) to a product (100% by mass) obtained by synthesis is preferably 90% by mass or more, more preferably 93% by mass, further preferably 97% by mass or more. When the content ratio of the compound (1) is 90% by mass or more, the trap of a carrier to an impurity level resulting from unintended impurities upon use of the compound (1) in a photoelectric conversion element can be more effectively and reliably avoided. As a result, a photoelectric conversion element having better performance can be obtained by suppressing carrier recombination. The content ratio can be measured by liquid chromatography, gas chromatography, elemental analysis, or the like, and a method known in the art can be used.

Hereinafter, preferred combinations of R₁, R₂, R₃, R₄, R₅ and R₆ in the compound (1) of the present embodiment in which both or any one of two X moieties is NR₆ will be shown. However, the compound (1) is not limited thereto.

| R₁ | R₂ | R₃ | R₄ | R₅ | R₆ |
|---|---|---|---|---|---|
| H | H | H | H | H | Ph |
| H | H | H | H | H | Py |
| H | H | H | H | H | p-CN—Ph |
| H | H | H | H | H | p-CF₂—Ph |
| H | H | H | H | H | p-MeO—Ph |
| H | H | H | H | H | 3,5-(CF₃)₂—Ph |
| H | H | H | H | H | 3.4-(CN)₂—Ph |
| H | H | H | H | H | 3-CF₃-4-CN—Ph |
| CN | H | H | H | H | Ph |
| CN | CN | H | H | H | Ph |
| H | CN | CN | H | H | Ph |
| CN | CN | CN | H | H | Ph |
| CN | CN | H | H | CN | Ph |
| CN | H | H | CN | CN | Ph |
| CN | H | H | H | H | Py |
| CN | CN | H | H | H | Py |
| H | CN | CN | H | H | Py |
| CN | CN | CN | H | H | Py |
| CN | CN | H | H | CN | Py |
| CN | H | H | CN | CN | Py |
| CN | H | H | H | H | p-CN—Ph |
| CN | CN | H | H | H | p-CN—Ph |
| H | CN | CN | H | H | p-CN—Ph |
| CN | CN | CN | H | H | p-CN—Ph |
| CN | CN | H | H | CN | p-CN—Ph |
| CN | H | H | CN | CN | p-CN—Ph |
| CN | H | H | H | H | p-CF₃—Ph |
| CN | CN | H | H | H | p-CF₃—Ph |
| H | CN | CN | H | H | p-CF₃—Ph |
| CN | CN | CN | H | H | p-CF₃—Ph |
| CN | CN | H | H | CN | p-CF₃—Ph |
| CN | H | H | CN | CN | p-CF₃—Ph |
| CN | H | H | H | H | p-MeO—Ph |
| CN | CN | H | H | H | p-MeO—Ph |
| H | CN | CN | H | H | p-MeO—Ph |
| CN | CN | CN | H | H | p-MeO—Ph |
| CN | CN | H | H | CN | p-MeO—Ph |
| CN | H | H | CN | CN | p-MeO—Ph |
| CN | H | H | H | H | 3,5-(CF₃)₂—Ph |
| CN | CN | H | H | H | 3,5-(CF₃)₂—Ph |
| H | CN | CN | H | H | 3,5-(CF₃)₂—Ph |
| CN | CN | CN | H | H | 3,5-(CF₃)₂—Ph |
| CN | CN | H | H | CN | 3,5-(CF₃)₂—Ph |
| CN | H | H | CN | CN | 3,5-(CF₃)₂—Ph |
| CN | H | H | H | H | 3,4-(CN)₂—Ph |
| CN | CN | H | H | H | 3,4-(CN)₂—Ph |
| H | CN | CN | H | H | 3,4-(CN)₂—Ph |
| CN | CN | CN | H | H | 3,4-(CN)₂—Ph |
| CN | CN | H | H | CN | 3,4-(CN)₂—Ph |
| CN | H | H | CN | CN | 3,4-(CN)₂—Ph |
| CN | H | H | H | H | 3-CF₃-4-CN—Ph |
| CN | CN | H | H | H | 3-CF₃-4-CN—Ph |
| H | CN | CN | H | H | 3-CF₃-4-CN—Ph |
| CN | CN | CN | H | H | 3-CF₃-4-CN—Ph |
| CN | CN | H | H | CN | 3-CF₃-4-CN—Ph |
| CN | H | H | CN | CN | 3-CF₃-4-CN—Ph |
| F | H | H | H | H | Ph |
| F | F | H | H | H | Ph |
| H | F | F | H | H | Ph |
| F | F | F | H | H | Ph |
| F | F | H | H | F | Ph |
| F | H | H | F | F | Ph |
| F | H | H | H | H | Py |
| F | F | H | H | H | Py |
| H | F | F | H | H | Py |
| F | F | F | H | H | Py |
| F | F | H | H | F | Py |
| F | H | H | F | F | Py |
| F | H | H | H | H | p-CN—Ph |
| F | F | H | H | H | p-CN—Ph |
| H | F | F | H | H | p-CN—Ph |
| F | F | F | H | H | p-CN—Ph |
| F | F | H | H | F | p-CN—Ph |
| F | H | H | F | F | p-CN—Ph |
| F | H | H | H | H | p-CF₃—Ph |
| F | F | H | H | H | p-CF₃—Ph |
| H | F | F | H | H | p-CF₃—Ph |
| F | F | F | H | H | p-CF₃—Ph |
| F | F | H | H | F | p-CF₃—Ph |
| F | H | H | F | F | p-CF₃—Ph |
| F | H | H | H | H | p-MeO—Ph |
| F | F | H | H | H | p-MeO—Ph |
| H | F | F | H | H | p-MeO—Ph |
| F | F | F | H | H | p-MeO—Ph |
| F | F | H | H | F | p-MeO—Ph |
| F | H | H | F | F | p-MeO—Ph |
| F | H | H | H | H | 3,5-(CF₃)₂—Ph |
| F | F | H | H | H | 3,5-(CF₃)₂—Ph |
| H | F | F | H | H | 3,5-(CF₃)₂—Ph |
| F | F | F | H | H | 3,5-(CF₃)₂—Ph |
| F | F | H | H | F | 3,5-(CF₃)₂—Ph |
| F | H | H | F | F | 3,5-(CF₃)₂—Ph |
| F | H | H | H | H | 3,4-(CN)₂—Ph |
| F | F | H | H | H | 3,4-(CN)₂—Ph |
| H | F | F | H | H | 3,4-(CN)₂—Ph |
| F | F | F | H | H | 3,4-(CN)₂—Ph |
| F | F | H | H | F | 3,4-(CN)₂—Ph |
| F | H | H | F | F | 3,4-(CN)₂—Ph |
| F | H | H | H | H | 3-CF₃-4-CN—Ph |
| F | F | H | H | H | 3-CF₃-4-CN—Ph |
| H | F | F | H | H | 3-CF₃-4-CN—Ph |
| F | F | F | H | H | 3-CF₃-4-CN—Ph |
| F | F | H | H | F | 3-CF₃-4-CN—Ph |
| F | H | H | F | F | 3-CF₃-4-CN—Ph |
| Cl | H | H | H | H | Ph |
| Cl | Cl | H | H | H | Ph |
| H | Cl | Cl | H | H | Ph |
| Cl | Cl | Cl | H | H | Ph |
| Cl | Cl | H | H | Cl | Ph |
| Cl | H | H | Cl | Cl | Ph |
| Cl | H | H | H | H | Py |
| Cl | Cl | H | H | H | Py |
| H | Cl | Cl | H | H | Py |
| Cl | Cl | Cl | H | H | Py |
| Cl | Cl | H | H | Cl | Py |
| Cl | H | H | Cl | Cl | Py |
| Cl | H | H | H | H | p-CN—Ph |
| Cl | Cl | H | H | H | p-CN—Ph |
| H | Cl | Cl | H | H | p-CN—Ph |
| Cl | Cl | Cl | H | H | p-CN—Ph |
| Cl | Cl | H | H | Cl | p-CN—Ph |
| Cl | H | H | Cl | Cl | p-CN—Ph |
| Cl | H | H | H | H | p-CF₃—Ph |
| Cl | Cl | H | H | H | p-CF₃—Ph |
| H | Cl | Cl | H | H | p-CF₃—Ph |
| Cl | Cl | Cl | H | H | p-CF₃—Ph |
| Cl | Cl | H | H | Cl | p-CF₃—Ph |
| Cl | H | H | Cl | Cl | p-CF₃—Ph |
| Cl | H | H | H | H | p-MeO—Ph |
| Cl | Cl | H | H | H | p-MeO—Ph |
| H | Cl | Cl | H | H | p-MeO—Ph |
| Cl | Cl | Cl | H | H | p-MeO—Ph |
| Cl | Cl | H | H | Cl | p-MeO—Ph |
| Cl | H | H | Cl | Cl | p-MeO—Ph |
| Cl | H | H | H | H | 3,5-(CF₃)₂—Ph |
| Cl | Cl | H | H | H | 3,5-(CF₃)₂—Ph |
| H | Cl | Cl | H | H | 3,5-(CF₃)₂—Ph |
| Cl | Cl | Cl | H | H | 3,5-(CF₃)₂—Ph |
| Cl | Cl | H | H | Cl | 3,5-(CF₃)₂—Ph |
| Cl | H | H | Cl | Cl | 3,5-(CF₃)₂—Ph |
| Cl | H | H | H | H | 3,4-(CN)₂—Ph |
| Cl | Cl | H | H | H | 3,4-(CN)₂—Ph |
| H | Cl | Cl | H | H | 3,4-(CN)₂—Ph |
| Cl | Cl | Cl | H | H | 3,4-(CN)₂—Ph |
| Cl | Cl | H | H | Cl | 3,4-(CN)₂—Ph |
| Cl | H | H | Cl | Cl | 3,4-(CN)₂—Ph |
| Cl | H | H | H | H | 3-CF₃-4-CN—Ph |
| Cl | Cl | H | H | H | 3-CF₃-4-CN—Ph |
| H | Cl | Cl | H | H | 3-CF₃-4-CN—Ph |
| Cl | Cl | Cl | H | H | 3-CF₃-4-CN—Ph |
| Cl | Cl | H | H | Cl | 3-CF₃-4-CN—Ph |
| Cl | H | H | Cl | Cl | 3-CF₃-4-CN—Ph |
| CF₃ | H | H | H | H | Ph |
| CF₃ | CF₃ | H | H | H | Ph |
| H | CF₃ | CF₃ | H | H | Ph |
| CF₃ | CF₃ | CF₃ | H | H | Ph |
| CF₃ | CF₃ | H | H | CF₃ | Ph |
| CF₃ | H | H | CF₃ | CF₃ | Ph |
| CF₃ | H | H | H | H | Py |
| CF₃ | CF₃ | H | H | H | Py |
| H | CF₃ | CF₃ | H | H | Py |
| CF₃ | CF₃ | CF₃ | H | H | Py |
| CF₃ | CF₃ | H | H | CF₃ | Py |
| CF₃ | H | H | CF₃ | CF₃ | Py |
| CF₃ | H | H | H | H | p-CN—Ph |
| CF₃ | CF₃ | H | H | H | p-CN—Ph |
| H | CF₃ | CF₃ | H | H | p-CN—Ph |
| CF₃ | CF₃ | CF₃ | H | H | p-CN—Ph |
| CF₃ | CF₃ | H | H | CF₃ | p-CN—Ph |
| CF₃ | H | H | CF₃ | CF₃ | p-CN—Ph |
| CF₃ | H | H | H | H | p-CF₃—Ph |
| CF₃ | CF₃ | H | H | H | p-CF₃—Ph |
| H | CF₃ | CF₃ | H | H | p-CF₃—Ph |
| CF₃ | CF₃ | CF₃ | H | H | p-CF₃—Ph |
| CF₃ | CF₃ | H | H | CF₃ | p-CF₃—Ph |
| CF₃ | H | H | CF₃ | CF₃ | p-CF₃—Ph |
| CF₃ | H | H | H | H | p-MeO—Ph |
| CF₃ | CF₃ | H | H | H | p-MeO—Ph |
| H | CF₃ | CF₃ | H | H | p-MeO—Ph |
| CF₃ | CF₃ | CF₃ | H | H | p-MeO—Ph |
| CF₃ | CF₃ | H | H | CF₃ | p-MeO—Ph |
| CF₃ | H | H | CF₃ | CF₃ | p-MeO—Ph |
| CF₃ | H | H | H | H | 3,5-(CF₃)₂—Ph |
| CF₃ | CF₃ | H | H | H | 3,5-(CF₃)₂—Ph |
| H | CF₃ | CF₃ | H | H | 3,5-(CF₃)₂—Ph |
| CF₃ | CF₃ | CF₃ | H | H | 3,5-(CF₃)₂—Ph |
| CF₃ | CF₃ | H | H | CF₃ | 3,5-(CF₃)₂—Ph |
| CF₃ | H | H | CF₃ | CF₃ | 3,5-(CF₃)₂—Ph |
| CF₃ | H | H | H | H | 3,4-(CN)₂—Ph |
| CF₃ | CF₃ | H | H | H | 3,4-(CN)₂—Ph |
| H | CF₃ | CF₃ | H | H | 3,4-(CN)₂—Ph |
| CF₃ | CF₃ | CF₃ | H | H | 3,4-(CN)₂—Ph |
| CF₃ | CF₃ | H | H | CF₃ | 3,4-(CN)₂—Ph |
| CF₃ | H | H | CF₃ | CF₃ | 3,4-(CN)₂—Ph |
| CF₃ | H | H | H | H | 3-CF₃-4-CN—Ph |
| CF₃ | CF₃ | H | H | H | 3-CF₃-4-CN—Ph |
| H | CF₃ | CF₃ | H | H | 3-CF₃-4-CN—Ph |
| CF₃ | CF₃ | CF₃ | H | H | 3-CF₃-4-CN—Ph |
| CF₃ | CF₃ | H | H | CF₃ | 3-CF₃-4-CN—Ph |
| CF₃ | H | H | CF₃ | CF₃ | 3-CF₃-4-CN—Ph |
| NO₂ | H | H | H | H | Ph |
| NO₂ | NO₂ | H | H | H | Ph |
| H | NO₂ | NO₂ | H | H | Ph |
| NO₂ | NO₂ | NO₂ | H | H | Ph |
| NO₂ | NO₂ | H | H | NO₂ | Ph |
| NO₂ | H | H | NO₂ | NO₂ | Ph |
| NO₂ | H | H | H | H | Py |
| NO₂ | NO₂ | H | H | H | Py |
| H | NO₂ | NO₂ | H | H | Py |
| NO₂ | NO₂ | NO₂ | H | H | Py |
| NO₂ | NO₂ | H | H | NO₂ | Py |
| NO₂ | H | H | NO₂ | NO₂ | Py |
| NO₂ | H | H | H | H | p-CN—Ph |
| NO₂ | NO₂ | H | H | H | p-CN—Ph |
| H | NO₂ | NO₂ | H | H | p-CN—Ph |
| NO₂ | NO₂ | NO₂ | H | H | p-CN—Ph |
| NO₂ | NO₂ | H | H | NO₂ | p-CN—Ph |
| NO₂ | H | H | NO₂ | NO₂ | p-CN—Ph |
| NO₂ | H | H | H | H | p-CF₃—Ph |
| NO₂ | NO₂ | H | H | H | p-CF₃—Ph |
| H | NO₂ | NO₂ | H | H | p-CF₃—Ph |
| NO₂ | NO₂ | NO₂ | H | H | p-CF₃—Ph |
| NO₂ | NO₂ | H | H | NO₂ | p-CF₃—Ph |
| NO₂ | H | H | NO₂ | NO₂ | p-CF₃—Ph |
| NO₂ | H | H | H | H | p-MeO—Ph |
| NO₂ | NO₂ | H | H | H | p-MeO—Ph |
| H | NO₂ | NO₂ | H | H | p-MeO—Ph |
| NO₂ | NO₂ | NO₂ | H | H | p-MeO—Ph |
| NO₂ | NO₂ | H | H | NO₂ | p-MeO—Ph |
| NO₂ | H | H | NO₂ | NO₂ | p-MeO—Ph |
| NO₂ | H | H | H | H | 3,5-(CF₃)₂—Ph |
| NO₂ | NO₂ | H | H | H | 3,5-(CF₃)₂—Ph |
| H | NO₂ | NO₂ | H | H | 3,5-(CF₃)₂—Ph |
| NO₂ | NO₂ | NO₂ | H | H | 3,5-(CF₃)₂—Ph |
| NO₂ | NO₂ | H | H | NO₂ | 3,5-(CF₃)₂—Ph |
| NO₂ | H | H | NO₂ | NO₂ | 3,5-(CF₃)₂—Ph |
| NO₂ | H | H | H | H | 3,4-(CN)₂—Ph |
| NO₂ | NO₂ | H | H | H | 3,4-(CN)₂—Ph |
| H | NO₂ | NO₂ | H | H | 3,4-(CN)₂—Ph |
| NO₂ | NO₂ | NO₂ | H | H | 3,4-(CN)₂—Ph |
| NO₂ | NO₂ | H | H | NO₂ | 3,4-(CN)₂—Ph |
| NO₂ | H | H | NO₂ | NO₂ | 3,4-(CN)₂—Ph |
| NO₂ | H | H | H | H | 3-CF₃-4-CN—Ph |
| NO₂ | NO₂ | H | H | H | 3-CF₃-4-CN—Ph |
| H | NO₂ | NO₂ | H | H | 3-CF₃-4-CN—Ph |
| NO₂ | NO₂ | NO₂ | H | H | 3-CF₃-4-CN—Ph |
| NO₂ | NO₂ | H | H | NO₂ | 3-CF₃-4-CN—Ph |
| NO₂ | H | H | NO₂ | NO₂ | 3-CF₃-4-CN—Ph |
| CH₃ | H | H | H | H | Ph |
| CH₃ | CH₃ | H | H | H | Ph |
| H | CH₃ | CH₃ | H | H | Ph |
| CH₃ | CH₃ | CH₃ | H | H | Ph |
| CH₃ | CH₃ | H | H | CH₃ | Ph |
| CH₃ | H | H | CH₃ | CH₃ | Ph |
| CH₃ | H | H | H | H | Py |
| CH₃ | CH₃ | H | H | H | Py |
| H | CH₃ | CH₃ | H | H | Py |
| CH₃ | CH₃ | CH₃ | H | H | Py |
| CH₃ | CH₃ | H | H | CH₃ | Py |
| CH₃ | H | H | CH₃ | CH₃ | Py |
| CH₃ | H | H | H | H | p-CN—Ph |
| CH₃ | CH₃ | H | H | H | p-CN—Ph |
| H | CH₃ | CH₃ | H | H | p-CN—Ph |
| CH₃ | CH₃ | CH₃ | H | H | p-CN—Ph |
| CH₃ | CH₃ | H | H | CH₃ | p-CN—Ph |
| CH₃ | H | H | CH₃ | CH₃ | p-CN—Ph |
| CH₃ | H | H | H | H | p-CF₃—Ph |
| CH₃ | CH₃ | H | H | H | p-CF₃—Ph |
| H | CH₃ | CH₃ | H | H | p-CF₃—Ph |
| CH₃ | CH₃ | CH₃ | H | H | p-CF₃—Ph |
| CH₃ | CH₃ | H | H | CH₃ | p-CF₃—Ph |
| CH₃ | H | H | CH₃ | CH₃ | p-CF₃—Ph |
| CH₃ | H | H | H | H | p-MeO—Ph |
| CH₃ | CH₃ | H | H | H | p-MeO—Ph |
| H | CH₃ | CH₃ | H | H | p-MeO—Ph |
| CH₃ | CH₃ | CH₃ | H | H | p-MeO—Ph |
| CH₃ | CH₃ | H | H | CH₃ | p-MeO—Ph |
| CH₃ | H | H | CH₃ | CH₃ | p-MeO—Ph |
| CH₃ | H | H | H | H | 3,5-(CF₃)₂—Ph |
| CH₃ | CH₃ | H | H | H | 3,5-(CF₃)₂—Ph |
| H | CH₃ | CH₃ | H | H | 3,5-(CF₃)₂—Ph |
| CH₃ | CH₃ | CH₃ | H | H | 3,5-(CF₃)₂—Ph |
| CH₃ | CH₃ | H | H | CH₃ | 3,5-(CF₃)₂—Ph |
| CH₃ | H | H | CH₃ | CH₃ | 3,5-(CF₃)₂—Ph |
| CH₃ | H | H | H | H | 3,4-(CN)₂—Ph |
| CH₃ | CH₃ | H | H | H | 3,4-(CN)₂—Ph |
| H | CH₃ | CH₃ | H | H | 3,4-(CN)₂—Ph |
| CH₃ | CH₃ | CH₃ | H | H | 3,4-(CN)₂—Ph |
| CH₃ | CH₃ | H | H | CH₃ | 3,4-(CN)₂—Ph |
| CH₃ | H | H | CH₃ | CH₃ | 3,4-(CN)₂—Ph |
| CH₃ | H | H | H | H | 3-CF₃-4-CN—Ph |
| CH₃ | CH₃ | H | H | H | 3-CF₃-4-CN—Ph |
| H | CH₃ | CH₃ | H | H | 3-CF₃-4-CN—Ph |
| CH₃ | CH₃ | CH₃ | H | H | 3-CF₃-4-CN—Ph |
| CH₃ | CH₃ | H | H | CH₃ | 3-CF₃-4-CN—Ph |
| CH₃ | H | H | CH₃ | CH₃ | 3-CF₃-4-CN—Ph |

Hereinafter, preferred combinations of R₁, R₂, R₃, R₄ and R₅ in the compound (1) of the present embodiment in which X is an oxygen atom will be shown. However, the compound (1) is not limited thereto.

| R₁ | R₂ | R₃ | R₄ | R₅ |
|---|---|---|---|---|
| H | H | H | H | H |
| CN | H | H | H | H |
| CN | CN | H | H | H |
| H | CN | CN | H | H |
| CN | CN | H | H | CN |
| CN | H | H | CN | CN |
| F | H | H | H | H |
| F | F | H | H | H |
| H | F | F | H | H |
| F | F | H | H | F |
| F | H | H | F | F |
| Cl | H | H | H | H |
| Cl | Cl | H | H | H |
| H | Cl | Cl | H | H |
| Cl | Cl | H | H | Cl |
| Cl | H | H | Cl | Cl |
| CF₃ | H | H | H | H |
| CF₃ | CF₃ | H | H | H |
| H | CF₃ | CF₃ | H | H |
| CF₃ | CF₃ | H | H | CF₃ |
| CF₃ | H | H | CF₃ | CF₃ |
| NO₂ | H | H | H | H |
| NO₂ | NO₂ | H | H | H |
| H | NO₂ | NO₂ | H | H |
| NO₂ | NO₂ | H | H | NO₂ |
| NO₂ | H | H | NO₂ | NO₂ |
| CH₃ | H | H | H | H |
| CH₃ | CH₃ | H | H | H |
| H | CH₃ | CH₃ | H | H |
| CH₃ | CH₃ | H | H | CH₃ |
| CH₃ | H | H | CH₃ | CH₃ |

Hereinafter, specific examples of the compound (1) will be shown. However, the compound (1) is not limited thereto.

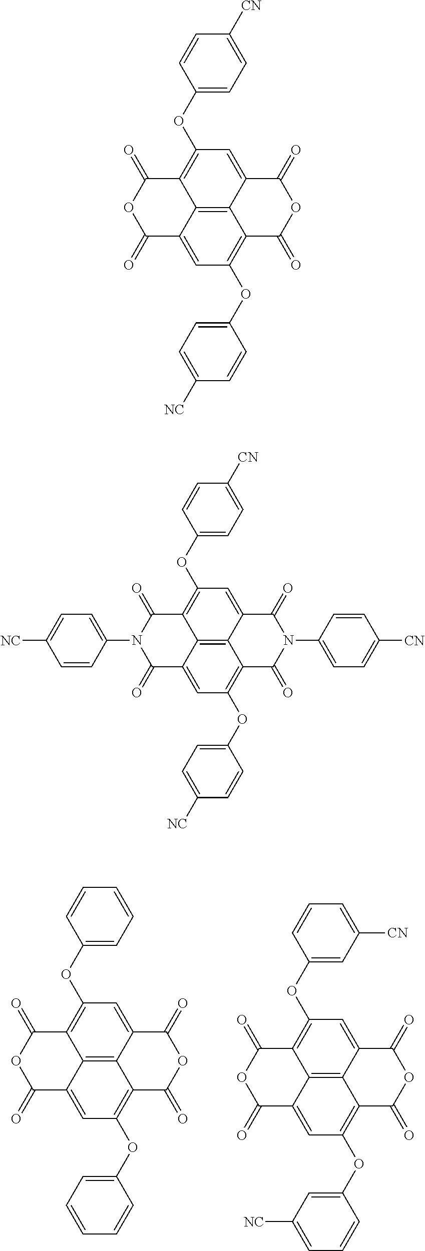 [SMILES: N#Cc1ccc(Oc2cc3c4c(c(Oc5ccc(C#N)cc5)cc5c4c2C(=O)OC5=O)C(=O)OC3=O)cc1 ; N#Cc1ccc(Oc2cc3c4c(c(Oc5ccc(C#N)cc5)cc5c4c2C(=O)N(c2ccc(C#N)cc2)C5=O)C(=O)N(c2ccc(C#N)cc2)C3=O)cc1 ; O=C1OC(=O)c2c(Oc3ccccc3)cc3c4c(c(Oc5ccccc5)cc1c24)C(=O)OC3=O ; N#Cc1cccc(Oc2cc3c4c(c(Oc5cccc(C#N)c5)cc5c4c2C(=O)OC5=O)C(=O)OC3=O)c1]

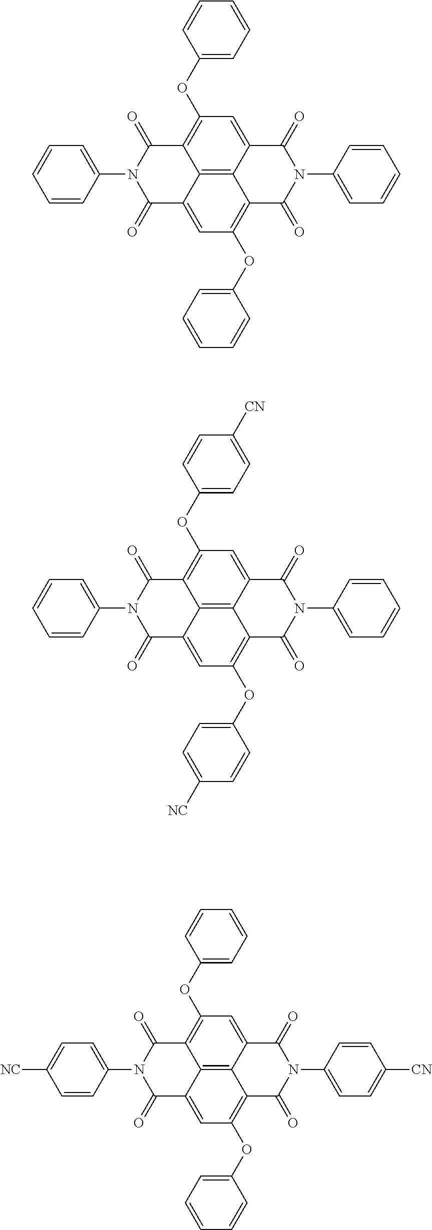 [SMILES: O=C1c2cc(Oc3ccccc3)c3c4c(cc(Oc5ccccc5)c(c24)C(=O)N1c1ccccc1)C(=O)N(c1ccccc1)C3=O ; N#Cc1ccc(Oc2cc3c4c(c(Oc5ccc(C#N)cc5)cc5c4c2C(=O)N(c2ccccc2)C5=O)C(=O)N(c2ccccc2)C3=O)cc1 ; N#Cc1ccc(N2C(=O)c3cc(Oc4ccccc4)c4c5c(cc(Oc6ccccc6)c(c35)C2=O)C(=O)N(c2ccc(C#N)cc2)C4=O)cc1]

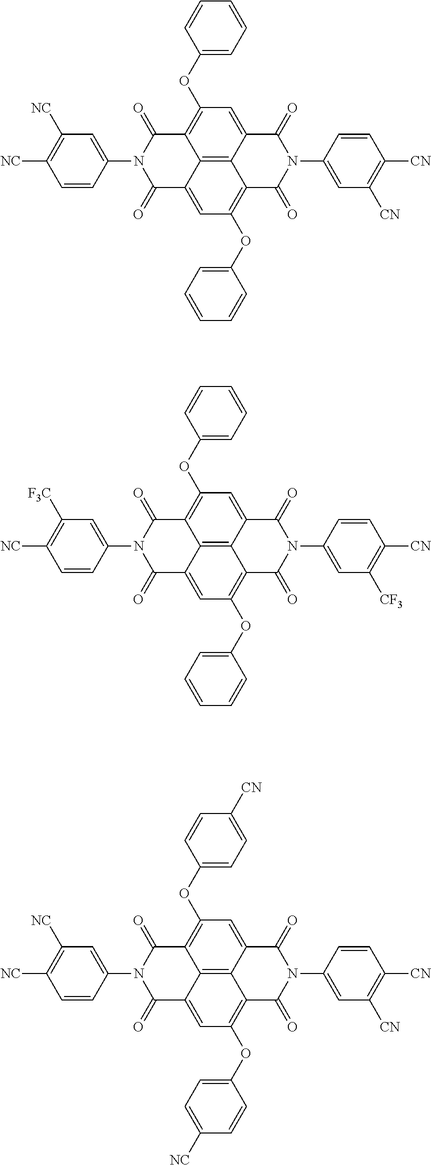 [SMILES: N#Cc1ccc(N2C(=O)c3cc(Oc4ccccc4)c4c5c(cc(Oc6ccccc6)c(c35)C2=O)C(=O)N(c2ccc(C#N)c(C#N)c2)C4=O)cc1C#N ; N#Cc1ccc(N2C(=O)c3cc(Oc4ccccc4)c4c5c(cc(Oc6ccccc6)c(c35)C2=O)C(=O)N(c2ccc(C#N)c(C(F)(F)F)c2)C4=O)cc1C(F)(F)F ; N#Cc1ccc(Oc2cc3c4c(c(Oc5ccc(C#N)cc5)cc5c4c2C(=O)N(c2ccc(C#N)c(C#N)c2)C5=O)C(=O)N(c2ccc(C#N)c(C#N)c2)C3=O)cc1]

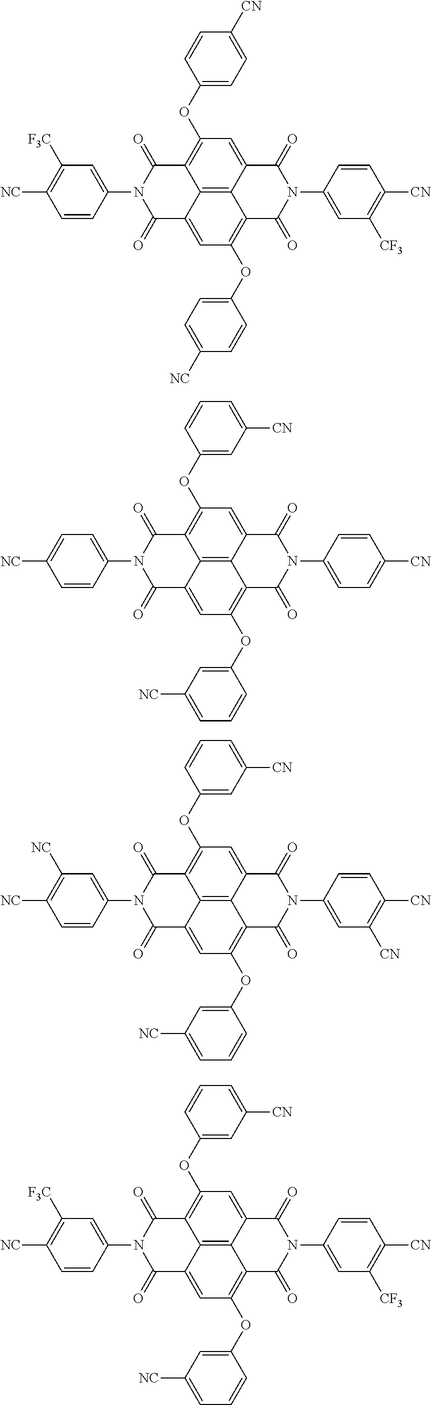

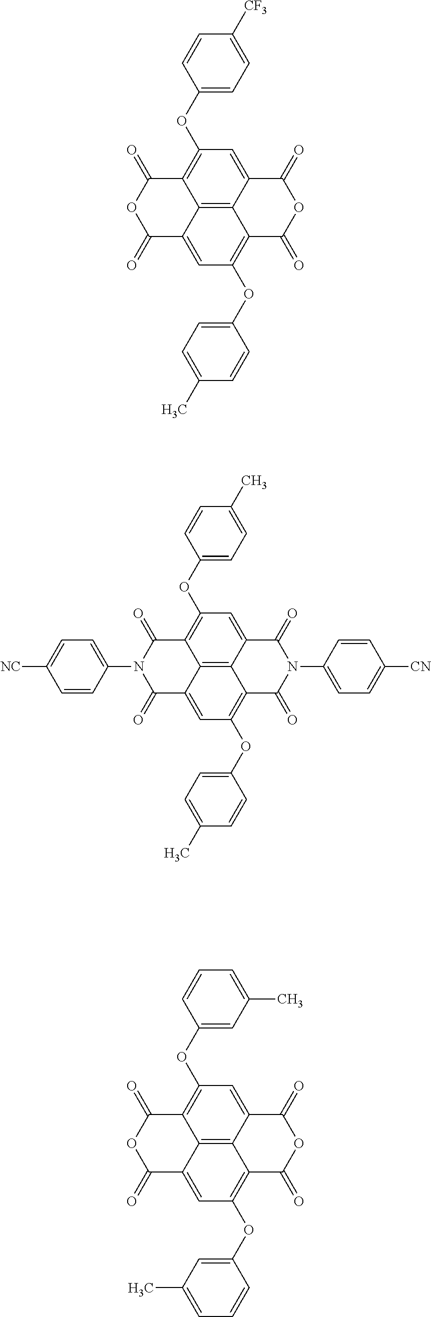 [SMILES: Cc1ccc(Oc2cc3c4c(c(Oc5ccc(C(F)(F)F)cc5)cc5c4c2C(=O)OC5=O)C(=O)OC3=O)cc1 ; Cc1ccc(Oc2cc3c4c(c(Oc5ccc(C)cc5)cc5c4c2C(=O)N(c2ccc(C#N)cc2)C5=O)C(=O)N(c2ccc(C#N)cc2)C3=O)cc1 ; Cc1cccc(Oc2cc3c4c(c(Oc5cccc(C)c5)cc5c4c2C(=O)OC5=O)C(=O)OC3=O)c1]

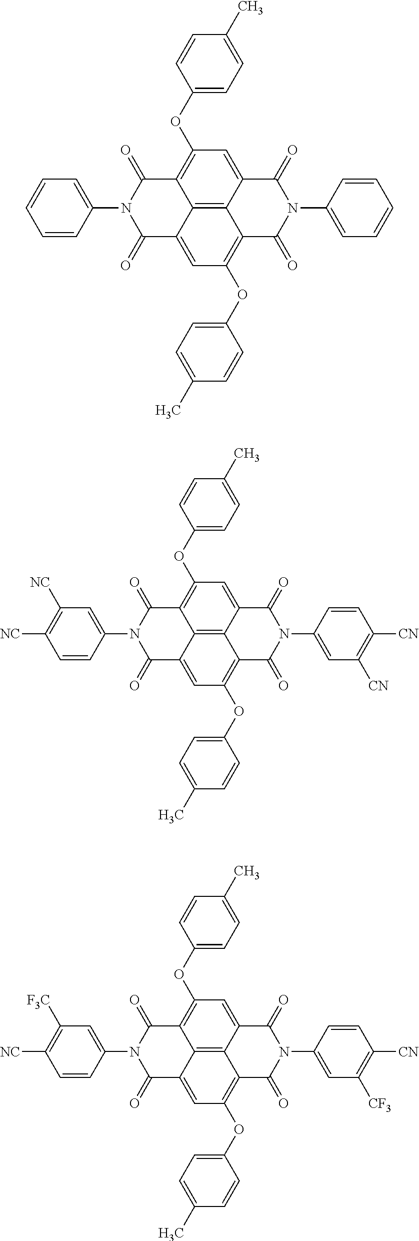 [SMILES: Cc1ccc(Oc2cc3c4c(c(Oc5ccc(C)cc5)cc5c4c2C(=O)N(c2ccccc2)C5=O)C(=O)N(c2ccccc2)C3=O)cc1 ; Cc1ccc(Oc2cc3c4c(c(Oc5ccc(C)cc5)cc5c4c2C(=O)N(c2ccc(C#N)c(C#N)c2)C5=O)C(=O)N(c2ccc(C#N)c(C#N)c2)C3=O)cc1 ; Cc1ccc(Oc2cc3c4c(c(Oc5ccc(C)cc5)cc5c4c2C(=O)N(c2ccc(C#N)c(C(F)(F)F)c2)C5=O)C(=O)N(c2ccc(C#N)c(C(F)(F)F)c2)C3=O)cc1]

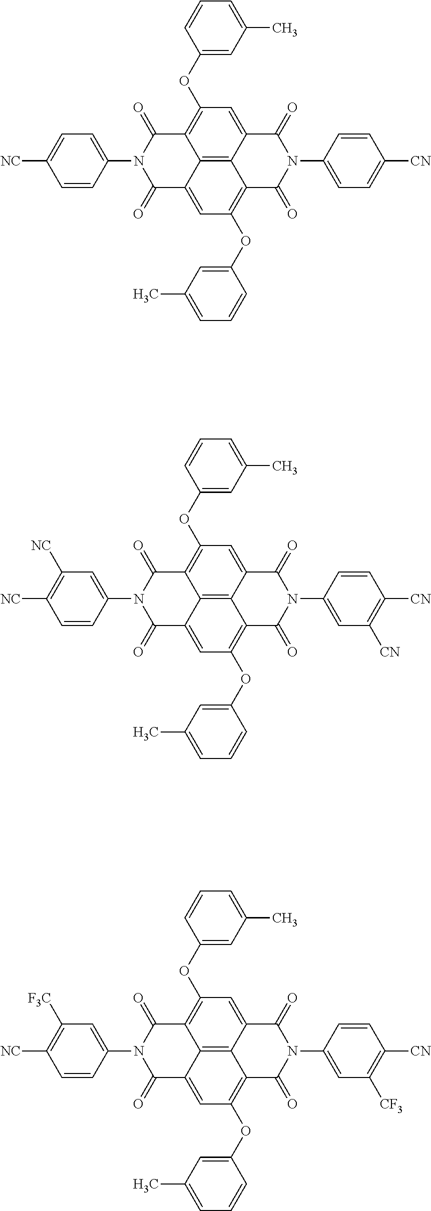 [SMILES: Cc1cccc(Oc2cc3c4c(c(Oc5cccc(C)c5)cc5c4c2C(=O)N(c2ccc(C#N)cc2)C5=O)C(=O)N(c2ccc(C#N)cc2)C3=O)c1 ; Cc1cccc(Oc2cc3c4c(c(Oc5cccc(C)c5)cc5c4c2C(=O)N(c2ccc(C#N)c(C#N)c2)C5=O)C(=O)N(c2ccc(C#N)c(C#N)c2)C3=O)c1 ; Cc1cccc(Oc2cc3c4c(c(Oc5cccc(C)c5)cc5c4c2C(=O)N(c2ccc(C#N)c(C(F)(F)F)c2)C5=O)C(=O)N(c2ccc(C#N)c(C(F)(F)F)c2)C3=O)c1]

The compound (1) of the present embodiment is particularly preferably a compound represented by the following formula:

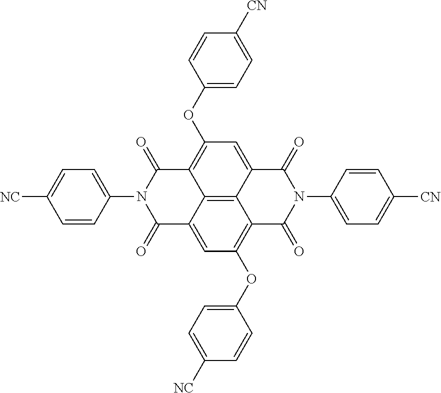 [SMILES: N#Cc1ccc(Oc2cc3c4c(c(Oc5ccc(C#N)cc5)cc5c4c2C(=O)N(c2ccc(C#N)cc2)C5=O)C(=O)N(c2ccc(C#N)cc2)C3=O)cc1]

Such a compound has four cyano groups and can thereby suppress leak current in the dark and exhibits excellent characteristics, particularly, as a photoelectric conversion element material. A reason for this is considered as follows: the energy level of the lowest unoccupied molecular orbital of such a compound is decreased, and the energy level of the highest occupied molecular orbital is also decreased. As a result, the compound has a high energy gap while keeping its low energy level of the lowest unoccupied molecular orbital. The compound described above can thereby suppress leak current in the dark and presumably produces excellent characteristics as a photoelectric conversion element material. However, the reason is not limited thereto

The compound (1) can be synthesized by, for example, the following scheme.

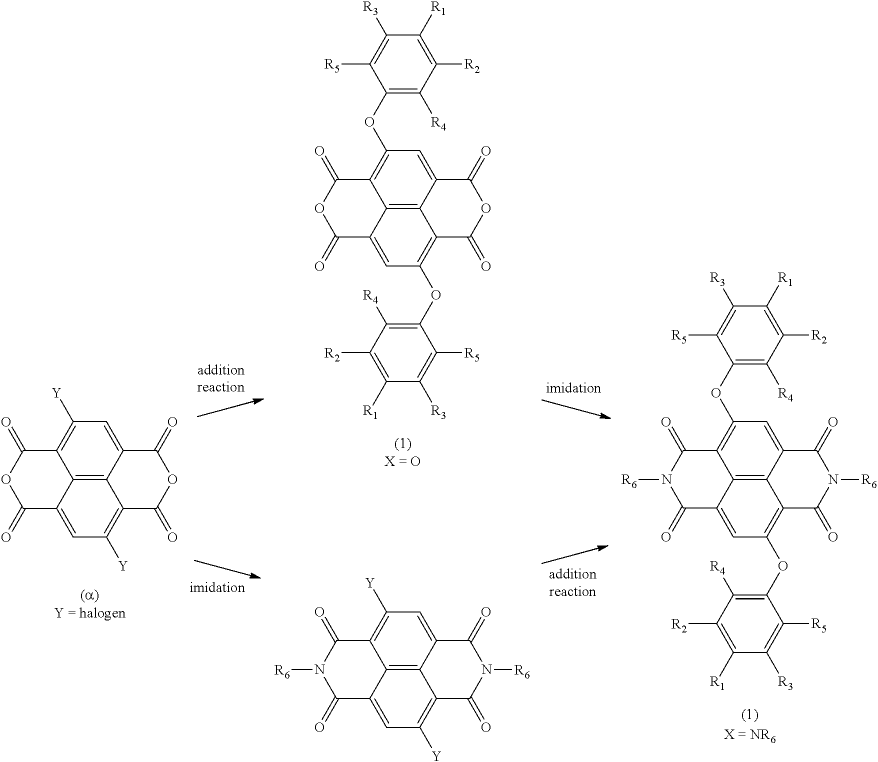

More specifically, the compound (1) in which X is an oxygen atom can be obtained through side chain addition reaction from, for example, a commercially available compound (α). The compound (1) in which X is NR₆ can be obtained by further performing imidation reaction. Alternatively, the compound (1) in which X is NR₆ can be obtained by performing side chain addition reaction through an intermediate obtained by the imidation of the commercially available compound (α). The compound of the formula (1) wherein one of two X moieties is an oxygen atom and the other moiety is NR₆ can be obtained by adjusting the equivalent of a reagent in imidation reaction. More specifically, the imidation can be synthesized by a method described in, for example, Organic Electronics, 63, 250 (2018). The side chain addition reaction can be performed using a compound in which R₁ to R₅ are introduced, or R₁ R₅ may be introduced after the side chain addition reaction. The side chain addition reaction can be pursued by coupling reaction or a method described in Asian Journal of Organic Chemistry, 2, 779 (2013).

### (Material for Photoelectric Conversion Element)

The compound (1) of the present embodiment is used as a photoelectric conversion element material. More specifically, the compound (1) is used as a material contained in each layer in a photoelectric conversion element mentioned later. Among such layers, the compound (1) is preferably contained in an auxiliary layer and more preferably contained in at least one of an electron transport layer and a hole blocking layer, from the viewpoint of more effectively and reliably exerting the working effects according to the present invention.

The compound (1) of the present embodiment can be directly used as a photosensitive material or may be mixed with an additional material and used as a photosensitive composition. The content of the compound (1) in the photosensitive composition may be 50% by mass or more based on the total amount of the composition. The content may be 95% by mass or less, may be 90% by mass or less, or may be 80% by mass or less. The material other than the compound (1) in the photosensitive composition is not particularly limited as long as the material can be contained in a usual photosensitive composition. Examples of such a material include n-type semiconductor materials, p-type semiconductor materials, and light-absorbing materials mentioned later. These materials can each be used singly or in combination of two or more thereof.

### (Organic Thin Film)

The organic thin film of the present embodiment contains the compound (1) of the present embodiment or the material for a photoelectric conversion element. Such an organic thin film can be prepared by a general dry film formation method or wet film formation method. Specific examples thereof include: resistance heating vapor deposition, electron beam vapor deposition, sputtering, and molecular lamination methods which are vacuum processes; casting which is a solution process; coating methods such as spin coating, dip coating, blade coating, wire bar coating, and spray coating; printing methods such as inkjet printing, screen printing, offset printing, and relief printing; and soft lithography approaches such as microcontact printing methods. It is generally desirable to be able to use the material for a photoelectric conversion element in a process of coating with the compound in a solution state, from the viewpoint of the easy processing. However, for a photoelectric conversion element as prepared by laminating organic thin films, a dry film formation method such as resistance heating vapor deposition is preferred because a coating solution might infiltrate into a film of a lower layer.

For example, in the dry film formation method, the material for a photoelectric conversion element of the present embodiment, and optionally, an additional material appropriate for the application of the photoelectric conversion element are mixed to prepare a composition, and the composition can then be vapor-deposited onto a base material or another film in vacuum to obtain an organic thin film. In the wet film formation method, the photoelectric conversion film of the present embodiment, and optionally, an additional material for the application of the photoelectric conversion element are mixed together with a solvent to prepare a liquid composition, with which a base material or another film can then be coated and printed, followed by drying to obtain an organic thin film.

The organic thin film of the present embodiment may contain a material other than the compound (1) serving as the material for a photoelectric conversion element of the present embodiment. The content of the compound (1) in the organic thin film of the present embodiment is not particularly limited as long as performance necessary for use as the material for a photoelectric conversion element is exerted. The content of the compound (1) may be, for example, 50% by mass or more based on the total amount of the organic thin film. The content is preferably 80% by mass or more, more preferably 90% by mass or more, further preferably 95% by mass or more, from the viewpoint of more effectively and reliably exerting the working effects according to the present invention. The upper limit of the content of the compound (1) may be 100% by mass. When the organic thin film of the present embodiment contains a material other than the compound (1), the material is not particularly limited as long as the material can be used as a usual material for a photoelectric conversion element. Examples of such a material include n-type semiconductor materials, p-type semiconductor materials, and light-absorbing materials mentioned later, molybdenum oxide called doping material, alkali metals, and alkali metal compounds. These materials can each be used singly or in combination of two or more thereof.

The thickness of the organic thin film depends on the resistance value and/or charge mobility of each substance and thus cannot be limited. The thickness is usually 0.5 nm or more and 5000 nm or less and may be 1 nm or more and 1000 nm or less or may be 5 nm or more and 500 nm or less.

The organic thin film of the present embodiment preferably has a local maximum absorption wavelength of an optical absorption band at 450 nm or less from the viewpoint of more effectively and reliably exerting the working effects according to the present invention.

### (Photoelectric Conversion Element)

The photoelectric conversion element of the present embodiment refers to an element that generates a charge in response to the quantity of an incident light, and outputs the generated charge to the outside of the photoelectric conversion element through a condenser (hereinafter, also referred to as an “accumulation unit”) for charge accumulation, a transistor circuit (hereinafter, also referred to as a “readout unit”) for readout, and the like. In this context, the photoelectric conversion element refers to an element having a photoelectric conversion film, which absorbs at least a portion of incident lights, disposed between a pair of opposed electrodes, and a light is incident on the photoelectric conversion element from above the electrodes. The photoelectric conversion film is a photosensitive thin film containing a material that absorbs at least a portion of incident lights in the infrared region, and generates holes and electrons as a result of light incidence. The photoelectric conversion element of the present embodiment may have a photoelectric conversion element which generates a charge in response to the quantity of an incident light in the infrared region (hereinafter, also referred to as an “infrared photoelectric conversion element”). In this context, the infrared photoelectric conversion element refers to an element having a photoelectric conversion film, which absorbs infrared light (hereinafter, also referred to as “infrared photoelectric conversion film”), disposed between a pair of opposed electrodes, and a light is incident on the infrared photoelectric conversion element from above the electrodes. The infrared photoelectric conversion film is a photosensitive thin film containing a material that absorbs at least a portion of incident lights in the infrared region (hereinafter, referred to as an “infrared absorptive material”), and generates holes and electrons as a result of light incidence.

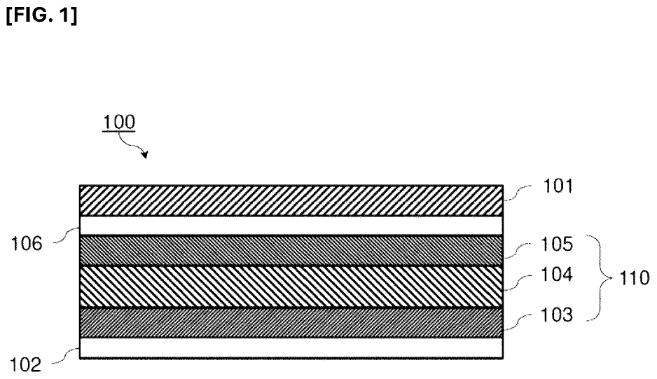

The photoelectric conversion element of the present embodiment will be described with appropriate reference to FIG. 1. A photoelectric conversion element 100 has a lower electrode 102 which is a first electrode film, an upper electrode 106 which is a second electrode film, and a photoelectric conversion film 110 positioned between the lower electrode 102 and the upper electrode 106. The photoelectric conversion element 100 may have a base material 101 which usually has insulation properties on the side, opposite to the photoelectric conversion film 110, of the upper electrode 106.

When the photoelectric conversion film 110 has hole-transporting properties or electron-transporting properties, the lower electrode 102 and the upper electrode 106 plays a role in extracting holes from the photoelectric conversion film 110 and collecting the holes, or extracting electrons from the photoelectric conversion film 110 and discharging the electrons. The material that may be used in these electrodes is not particularly limited as long as it has conductivity to some extent. The material is preferably selected in consideration of close contact with the adjacent photoelectric conversion film 110, electron affinity, an ionization potential and stability, etc. Examples of the material that may be used in the electrode include: conductive metal oxides such as tin oxide (NESA), indium oxide, indium tin oxide (ITO) and indium zinc oxide (IZO); metals such as gold, silver, platinum, chromium, aluminum, iron, cobalt, nickel and tungsten: inorganic conductive substances such as copper iodide and copper sulfide; conductive polymers such as polythiophene, polypyrrole and polyaniline; and carbon. These materials may each be used singly, may be used as a mixture of two or more thereof.

The lower electrode 102 which is the first electrode film is made of a conductive film having light permeability and made of, for example, indium tin oxide (ITO). The material constituting the lower electrode 102 is not limited to ITO. Examples thereof include tin oxide (SnO₂) materials supplemented with a dopant, and zinc oxide materials obtained by adding a dopant to zinc oxide (ZnO). Examples of the zinc oxide material include aluminum zinc oxide (AZO) obtained by adding aluminum (Al) as a dopant, gallium zinc oxide (GZO) obtained by adding gallium (Ga) thereas, and indium zinc oxide (IZO) obtained by adding indium (In) thereas. Alternative examples of the material constituting the lower electrode 102 also include CuI, InSbO₄, ZnMgO, CuInO₂, MgIN₂O₄, CdO, and ZnSnO₃. The thickness of the lower electrode 102 is, for example, 5 nm or more and 3000 nm or less and may be 5 nm or more and 500 nm or less or may be 10 nm or more and 300 nm or less.

The upper electrode 106 which is the second electrode film may be constituted by the same conductive film having light permeability as that of the lower electrode 102, or may be constituted by a metal, such as aluminum, which is usually used in an electrode in the photoelectric conversion element. In a solid-state imaging apparatus using a solid image sensor as one pixel, this upper electrode 106 may be isolated on a pixel basis or may be formed as a common electrode among the respective pixels. The thickness of the upper electrode 106 is, for example, 5 nm or more and 3000 nm or less and may be 5 nm or more and 500 nm or less or may be 10 nm or more and 300 nm or less.

The conductivity of the material for use in the electrodes such as the first electrode film and the second electrode film is not particularly limited as long as it does not hinder the light reception of the photoelectric conversion element more than necessary. The conductivity is preferably as high as possible from the viewpoint of the signal intensity and power consumption of the photoelectric conversion element. For example, as for a transparent electrode, an ITO film having conductivity equal to or less than a sheet resistance value of 300 ohms per square sufficiently functions as the electrode. However, a commercially available product of a substrate having an ITO film having conductivity on the order of several ohms per square (e.g., 5 to 9 ohms per square) is also obtainable, and such a substrate having high conductivity is desirable.

In the case of using an ITO film, the thickness of the electrode can be arbitrarily selected in consideration of conductivity and is usually 5 nm or more and 3000 nm or less, preferably 10 nm or more and 300 nm or less. Examples of the method for forming the film such as ITO include vapor deposition methods, electron beam methods, sputtering methods, chemical reaction methods and coating methods heretofore known in the art. The ITO film disposed on the substrate may be subjected, if necessary, to UV-ozone treatment or plasma treatment.

In the case of laminating a plurality of photoelectric conversion films differing in wavelength to be detected, the film of an electrode for use between the respective photoelectric conversion films needs to be transmissive to a light having a wavelength other than the lights to be detected by the respective photoelectric conversion films. From such a viewpoint, a material transmissive to 90% more of an incident light is preferably used in the film of the electrode, and a material transmissive to 95% or more of a light is more preferably used. The film of the electrode is the film of an electrode other than the pair of electrodes described above.

In the case of further establishing a visible photoelectric conversion unit that senses an infrared light or lights in different visible light regions beneath the photoelectric conversion element according to the present embodiment, the electrode for use in the photoelectric conversion element preferably has a transmittance of 90% or more, more preferably 95% or more, to the visible lights and the infrared light.

The material for the electrode that satisfies such conditions is preferably transparent conducting oxide (TCO) having a high transmittance to visible light and infrared light and a small resistance value. Although a thin film of a metal such as gold can be used as an electrode, its resistance value is extremely increased if the transmittance is adjusted to 90% or more. Thus, TCO is preferred for the electrode. The TCO is particularly preferably ITO, IZO, AZO, FTO, SnO₂, TiO₂ or ZnO₂.

The method for forming the electrode is not particularly limited and can be appropriately selected in consideration of aptitude for the electrode material. In the case of using a transparent electrode, specific examples of the formation method therefor include wet schemes such as printing schemes and coating schemes, physical schemes such as vacuum vapor deposition methods, sputtering methods and ion plating methods, and chemical schemes such as CVD and plasma CVD. When the electrode material is transparent conducting metal oxide such as ITO, examples of the formation method therefor include electron beam methods, sputtering methods, resistance heating vapor deposition methods, chemical reaction methods (e.g., a sol-gel method), and methods of coating with a dispersion of the metal oxide. The film of transparent conducting metal oxide such as ITO may be further subjected to UV-ozone treatment and plasma treatment.

The photoelectric conversion film 110 may contain the material for a photoelectric conversion element of the present embodiment or may include the organic thin film described above. More specifically, the photoelectric conversion film 110 has, for example, a photoelectric conversion layer 104, a first auxiliary layer 103 positioned on the lower electrode film 102 side of the photoelectric conversion layer 104, and a second auxiliary layer 105 positioned on the upper electrode film 106 side of the photoelectric conversion layer 104. The photoelectric conversion film 110 shown in FIG. 1 has the first auxiliary layer 103 and the second auxiliary layer 105, while the photoelectric conversion film may have only one of these auxiliary layers. Alternatively, the photoelectric conversion film may have neither of these auxiliary layers and have only the photoelectric conversion layer 104. When the photoelectric conversion film has no auxiliary layer, the photoelectric conversion layer 104 is the organic thin film described above. When the photoelectric conversion film has an auxiliary layer, at least one of the photoelectric conversion layer 104 and the auxiliary layer is the organic thin film described above. However, the auxiliary layer is preferably the organic thin film containing the material for a photoelectric conversion element of the present embodiment from the viewpoint of more effectively and reliably exerting the working effects according to the present invention.

The photoelectric conversion layer 104 may be an organic semiconductor film generally used as a photoelectric conversion layer or may be the organic thin film described above. In the photoelectric conversion layer 110, the organic semiconductor film or the organic thin film may be one layer or a plurality of layers. In the case of one layer, p-type organic semiconductor film, a n-type organic semiconductor film, or a mixed film thereof (hereinafter, which may be “bulk-hetero structures”) is used. On the other hand, in the case of a plurality of layers, the number of layers may be on the order of 2 to 10 layers. A structure is used where p-type organic semiconductor films, n-type organic semiconductor films, or mixed films thereof (hereinafter, which may be “bulk-hetero structures”) are laminated. A buffer layer may be inserted between the layers.

The photoelectric conversion layer 104 according to the present embodiment may or may not contain the material for a photoelectric conversion element of the present embodiment and may contain a material other than the material for a photoelectric conversion element of the present embodiment. Among others, the photoelectric conversion layer 104 preferably includes at least one or more of organic p-type semiconductor, organic n-type semiconductor, and light-absorbing materials because incident light energy with a desired wavelength can be more efficiently converted to electric signals. Among others, an organic p-type semiconductor that easily donates electrons (i.e., having a small ionization potential) to the light-absorbing material, or an organic n-type semiconductor that easily accepts electrons therefrom (i.e., having large electron affinity) is preferred because incident light energy can be still more efficiently converted to electric signals. In this context, the ionization potential (HOMO level) refers to a value measured by photoemission yield spectroscopy or photoelectron spectroscopy. The electron affinity (LUMO level) refers to a value determined by calculating an energy band gap value from the absorption end of the longest wavelength of near-infrared spectra, and subtracting the value from the HOMO level, or a value measured by inverse photoemission spectroscopy.

In the case of using an organic semiconductor film, the film may be one layer or may be two or more layers. The organic semiconductor film may be an organic p-type semiconductor film, may be an organic n-type semiconductor film, may be a light-absorbing material film, or may be a mixed film thereof (bulk-hetero structure). Particularly, the organic semiconductor film preferably has a layer having a bulk-hetero junction structure. In such a case, the photoelectric conversion film is allowed to have a bulk-hetero junction structure. This can compensate for the disadvantage, i.e., a short carrier diffusion length, of the photoelectric conversion film and improve photoelectric conversion efficiency.

The thickness of the photoelectric conversion layer 104 may be, for example, 0.5 nm or more and 5000 nm or less, may be 1 nm or more and 1000 nm or less, or may be 5 nm or more and 500 nm or less.

Hereinafter, the organic semiconductor will be described in detail.

The organic p-type semiconductor (compound) is a donor organic semiconductor (hereinafter, also referred to as a “donor organic compound”) and refers to an organic compound that is typified mainly by a hole-transporting organic compound and has a property of easily donating electrons. Further specifically, this compound refers to an organic compound having a smaller ionization potential when two organic materials are used in contact. Thus, any organic compound may be used as the donor organic compound as long as the organic compound has electron-donating properties.

Examples of such a donor organic compound include triarylamine compounds, benzidine compounds, pyrazoline compounds, styrylamine compounds, hydrazone compounds, triphenylmethane compounds, carbazole compounds, polysilane compounds, thiophene compounds, phthalocyanine compounds, cyanine compounds, merocyanine compounds, oxonol compounds, polyamine compounds, indole compounds, pyrrole compounds, pyrazole compounds, polyarylene compounds, condensed aromatic carbocyclic compounds (e.g., naphthalene derivatives, anthracene derivatives, phenanthrene derivatives, tetracene derivatives, pyrene derivatives, perylene derivatives, and fluoranthene derivatives), and metal complexes having nitrogen-containing heterocyclic compounds as ligands. The donor organic compound is not limited thereto, and as described above, an organic compound having a smaller ionization potential than that of an organic compound used as an acceptor organic compound may be used as the donor organic semiconductor.

The organic n-type semiconductor (compound) is an acceptor organic semiconductor (hereinafter, also referred to as an “acceptor organic compound”) and refers to an organic compound that is typified mainly by an electron-transporting organic compound and has a property of easily accepting electrons. Further specifically, this compound refers to an organic compound having larger electron affinity when two organic compounds are used in contact. Thus, any organic compound may be used as the acceptor organic compound as long as the organic compound has electron-accepting properties.

Examples of such an acceptor organic compound include condensed aromatic carbocyclic compounds (e.g., naphthalene derivatives, anthracene derivatives, phenanthrene derivatives, tetracene derivatives, pyrene derivatives, perylene derivatives, fluoranthene derivatives, and fullerene derivatives), 5- to 7-membered heterocyclic compounds containing nitrogen atom(s), oxygen atom(s), or sulfur atom(s) (e.g., pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, oxazole, indazole, benzimidazole, benzotriazole, benzoxazole, benzothiazole, carbazole, purine, triazolopyridazine, triazolopyrimidine, tetrazaindene, oxadiazole, imidazopyridine, pyralidine, pyrrolopyridine, thiadiazolopyridine, dibenzazepine, and tribenzazepine), polyarylene compounds, fluorene compounds, cyclopentadiene compounds, silyl compounds, and metal complexes having nitrogen-containing heterocyclic compounds as ligands. The acceptor organic compound is not limited thereto, and as described above, an organic compound having larger electron affinity than that of an organic compound used as a donor organic compound may be used as the acceptor organic semiconductor.

The light-absorbing material is a compound having a local maximum optical absorption wavelength in the visible light region, particularly, in the range of 450 nm or more and 650 nm or less. The absorption intensity in the local maximum optical absorption wavelength of the light-absorbing material is desirably larger than that in the local maximum optical absorption wavelength of a donor organic compound or an acceptor organic compound. The light-absorbing material having such absorption intensity can selectively absorb an incident light at the local maximum optical absorption wavelength. After an incident light is absorbed to the light-absorbing material so that photons become excitons, hole and electron carriers can be efficiently generated by causing exciton separation at the interface between a donor organic compound and an acceptor organic compound.

A compound generally called color can be used as such a light-absorbing material. Examples thereof include phthalocyanine derivatives, subphthalocyanine derivatives, quinacridone derivatives, porphyrin derivatives, naphthalene or perylene derivatives, phthaloperylene derivatives, styryl derivatives, cyanine derivatives, hemicyanine derivatives, merocyanine derivatives, rhodacyanine derivatives, oxonol derivatives, hemioxonol derivatives, croconium derivatives, squarylium derivatives, azamethine derivatives, arylidene derivatives, azo derivatives, azomethine derivatives, metallocene derivatives, fulgide derivatives, phenazine derivatives, phenothiazine derivatives, polyene derivatives, acridine derivatives, acridinone derivatives, diphenylamine derivatives, triarylamine derivatives such as triphenylamine, naphthylamine and styrylamine, quinophthalone derivatives, phenoxazine derivatives, chlorophyll derivatives, rhodamine derivatives, diphenylmethane or triphenylmethane derivatives, xanthene derivatives, acridine derivatives, phenoxazine derivatives, quinoline derivatives, oxazine derivatives, thiazine derivatives, quinone derivatives, benzoquinone derivatives, naphthoquinone derivatives, anthraquinone derivatives, indigo or thioindigo derivatives, pyrrole derivatives, pyridine derivatives, dipyrrin derivatives, indole derivatives, diketopyrrolopyrrole derivatives, coumarin derivatives, fluorene derivatives, fluorenone derivatives, fluoranthene derivatives, anthracene derivatives, pyrene derivatives, carbazole derivatives, phenylenediamine derivatives, benzidine derivatives, phenanthroline derivatives, imidazole derivatives, oxazoline derivatives, thiazoline derivatives, triazole derivatives, thiadiazole derivatives, oxazole derivatives, thiazole derivatives, oxadiazole derivatives, thiophene derivatives, selenophene derivatives, silole derivatives, germole derivatives, stilbene derivatives, phenylene vinylene derivatives, pentacene derivatives, rubrene derivatives, thienothiophene derivatives, benzodithiophene derivatives, xanthenoxanthene derivatives, and fullerene derivatives. The light-absorbing material is not limited thereto, and a compound having larger absorption intensity than that in the local maximum optical absorption wavelength of a donor organic compound or an acceptor organic compound can be used as the light-absorbing material, as described above. The light-absorbing material can also play a role as a donor organic compound or an acceptor organic compound.

The first auxiliary layer 103 has, for example, at least one of a hole blocking layer and an electron transport layer. When the first auxiliary layer 103 has two of these layers, the electron transport layer and the hole blocking layer are usually laminated in this order from the photoelectric conversion layer 104 side. The electron transport layer plays a role in transporting electrons generated in the photoelectric conversion layer 104 to the first electrode 102, and a role in blocking hole migration to the photoelectric conversion layer 104 from the first electrode 102 to which electrons are transported. The hole blocking layer plays a role in blocking hole migration from the first electrode 102 to the photoelectric conversion layer 104, preventing hole recombination in the photoelectric conversion layer 104, reducing dark current, reducing noise, and expanding a dynamic range. One layer may have the functions of both the hole blocking layer and the electron transport layer.

The second auxiliary layer 105 has, for example, at least one of an electron blocking layer and a hole transport layer. When the second auxiliary layer 105 has two of these layers, the hole transport layer and the electron blocking layer are usually layered in this order from the photoelectric conversion layer 104 side. The hole transport layer plays a role in transporting generated holes from the photoelectric conversion layer 104 to the second electrode 106, and a role in blocking electron migration to the photoelectric conversion layer 104 from the second electrode 106 to which holes are transported. The electron blocking layer plays a role in blocking electron migration from the second electrode 106 to the photoelectric conversion layer 104, preventing electron recombination in the photoelectric conversion layer 104, reducing dark current, reducing noise, and expanding a dynamic range. One layer may have the functions of both the electron blocking layer and the hole transport layer.

The material for a photoelectric conversion element of the present embodiment can be contained in both the first auxiliary layer 103 and the second auxiliary layer 105 and is preferably contained in the first auxiliary layer 103. In the photoelectric conversion element of the present embodiment, of the first auxiliary layer 103 and the second auxiliary layer 105, the first auxiliary layer 103 preferably includes the organic thin film. The material for a photoelectric conversion element of the present embodiment is more preferably contained in at least one of the hole blocking layer and the electron transport layer in the first auxiliary layer 103. In the photoelectric conversion element of the present embodiment, at least one of the hole blocking layer and the electron transport layer is preferably the organic thin film. These can more effectively and reliably exert the working effects according to the present invention.

Hereinafter, the material, other than the material for a photoelectric conversion element of the present embodiment, which may be contained in each layer in the auxiliary layers will be described.

The material for the hole transport layer is not particularly limited as long as it is known as a hole transport layer for photoelectric conversion elements such as solid image sensors. Examples thereof include polyaniline and doped materials thereof, and cyanogen compounds described in International Publication No. WO 2006/019270.

More specific examples of the material constituting the hole transport layer include selenium, iodides such as copper iodide (CuI), cobalt complexes such as layered cobalt oxide, CuSCN, molybdenum oxide (MoO₃, etc.), nickel oxide (NiO, etc.), 4CuBr·3S(C₄H₉) and organic hole transport materials. Among them, examples of the iodide include copper iodide (CuI). Examples of the layered cobalt oxide include AxCoO₂ (wherein A represents Li, Na, K, Ca, Sr or Ba, and 0≤X≤1). Examples of the organic hole transport material include polythiophene derivatives such as poly-3-hexylthiophene (P3HT) and poly(3,4-ethylenedioxythiophene) (PEDOT; e.g., trade name “BaytronP” manufactured by H.C. Starck-V Tech Ltd.), fluorene derivatives such as 2,2′,7,7′-tetrakis-(N,N-di-p-methoxyphenylamine)-9,9′-spirobifluorene (spiro-MeO-TAD), carbazole derivatives such as polyvinylcarbazole, triphenylamine derivatives, diphenylamine derivatives, polysilane derivatives, and polyaniline derivatives. Further examples of the material for the hole transport layer include compound semiconductors having monovalent copper, such as CuInSe₂ and copper sulfide (CuS), gallium phosphide (GaP), nickel oxide (NiO), cobalt oxide (CoO), iron oxide (FeO), bismuth oxide (Bi₂O₃), molybdenum oxide (MoO₂), and chromium oxide (Cr₂O₃).

The hole transport layer having a higher LUMO level than that of the photoelectric conversion film is preferred because an electron blocking function having a rectifying effect of suppressing the migration of electrons generated in the photoelectric conversion film to the electrode side is imparted thereto. Such a hole transport layer is also called an electron blocking layer.

Examples of the low-molecular organic compound as the material constituting the electron blocking layer include aromatic diamine compounds such as N,N′-bis(3-methylphenyl)-(1,1′-biphenyl)-4,4′-diamine (TPD) and 4,4′-bis[N-(naphthyl)-N-phenyl-amino]biphenyl (α-NPD), oxazole, oxadiazole, triazole, imidazole, imidazolone, stilbene derivatives, pyrazoline derivatives, tetrahydroimidazole, polyarylalkane, butadiene, 4,4′,4″-tris(N-(3-methylphenyl)N-phenylamino)triphenylamine (m-MTDATA), porphyrin compounds such as porphyrin, copper tetraphenylporphyrin, phthalocyanine, copper phthalocyanine and titanium phthalocyanine oxide, triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, and silazanes derivatives. Examples of the high-molecular organic compound include polymers such as phenylene vinylene, fluorene, carbazole, indole, pyrene, pyrrole, picoline, thiophene, acetylene and diacetylene, and derivatives thereof. A compound having sufficient hole-transporting properties, albeit being not an electron-donating compound, may be used as the material constituting the electron blocking layer. Examples of the inorganic compound as the material constituting the electron blocking layer include metal oxides such as calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide and iridium oxide, selenium, tellurium and antimony sulfide. These materials may each be used singly or in combination of two or more thereof.

The thickness of the hole transport layer is preferably 10 nm or more and 300 nm or less, more preferably 30 nm or more and 250 nm or less, further preferably 50 nm or more and 200 nm or less, from the viewpoint of suppressing dark current, and preventing reduction in photoelectric conversion efficiency.

The method for forming the hole transport layer and the electron blocking layer may be a heretofore known method and may be any of dry film formation methods such as vacuum vapor deposition methods, and wet film formation methods such as solution coating methods. A wet film formation method is preferred from the viewpoint of being able to level a coated surface. Examples of the dry film formation method include vapor deposition methods such as vacuum vapor deposition methods, and sputter methods. The vapor deposition may be any of physical vapor deposition (PVD) and chemical vapor deposition (CVD) and is preferably physical vapor deposition such as vacuum vapor deposition. Examples of the wet film formation method include inkjet methods, spray methods, nozzle print methods, spin coating methods, dip coating methods, casting methods, die coating methods, roll coating methods, bar coating methods and gravure coating methods.

The material constituting the electron transport layer is not particularly limited as long as it is known as an electron transport layer for photoelectric conversion elements such as solid image sensors. Examples thereof include organic compounds such as perfluoro forms (e.g., perfluoropentacene, perfluorophthalocyanine, etc.) of octaazaporphyrin and p-type semiconductors, fullerene, fullerene derivatives (e.g., [6,6]-phenyl-C61-butyric acid methyl ester; PCBM, etc.), perylene, indenoindene and indenoindene derivatives, and inorganic oxides such as titanium oxide (TiO₂, etc.), nickel oxide (NiO), tin oxide (SnO₂), tungsten oxide (WO₂, WO₃, W₂O₃, etc.), zinc oxide (ZnO), niobium oxide (Nb₂O₅, etc.), tantalum oxide (Ta₂O₅, etc.), yttrium oxide (Y₂O₃, etc.), and strontium titanate (SrTiO₃, etc.). The electron transport layer may be a porous layer or may be a dense layer. In the case of laminating them, the porous electron transport layer and the dense electron transport layer are preferably laminated and disposed in this order from the photoelectric conversion film side.

The electron transport layer having a lower HOMO level than that of the photoelectric conversion film is preferred because a hole blocking function having a rectifying effect of suppressing the migration of holes generated in the photoelectric conversion film to the opposite electrode side is imparted thereto. Such an electron transport layer is also called a hole blocking layer.

Examples of the material constituting the hole blocking layer include oxadiazole derivatives such as 1,3-bis(4-tert-butylphenyl-1,3,4-oxadiazolyl)phenylene (OXD-7), anthraquinone dimethane derivatives, diphenylquinone derivatives, bathocuproine, bathophenanthroline, and their derivatives, triazine compounds, triazole compounds, tris(8-hydroxyquinolinato)aluminum complexes, bis(4-methyl-8-quinolinato)aluminum complexes, silole compounds, porphyrin compounds, styryl compounds such as DCM (4-dicyanomethylene-2-methyl-6-(4-(dimethylaminostyryl))-4H-pyran), n-type semiconductor materials such as naphthalenetetracarboxylic anhydride (NTCDA), naphthalenetetracarboxylic acid diimide, perylenetetracarboxylic anhydride (PTCDA), and perylenetetracarboxylic acid diimide, n-type inorganic oxides such as titanium oxide, zinc oxide and gallium oxide, and alkali metal fluorides such as lithium fluoride, sodium fluoride and cesium fluoride. Further, an organic semiconductor molecule doped with an alkali metal compound is also preferred because of having a function of improving electric junction to the opposite electrode. These materials may each be used singly or in combination of two or more thereof.

The thickness of the electron transport layer is preferably 10 nm or more and 300 nm or less, more preferably 30 nm or more and 250 nm or less, further preferably 50 nm or more and 200 nm or less, from the viewpoint of suppressing dark current, and preventing reduction in photoelectric conversion efficiency.

The method for forming the electron transport layer and the hole blocking layer may be a heretofore known method and may be any of dry film formation methods such as vacuum vapor deposition methods, and wet film formation methods such as solution coating methods. A wet film formation method is preferred from the viewpoint of being able to level a coated surface. Examples of the dry film formation method include vapor deposition methods such as vacuum vapor deposition methods, and sputter methods. The vapor deposition may be any of physical vapor deposition (PVD) and chemical vapor deposition (CVD) and is preferably physical vapor deposition such as vacuum vapor deposition. Examples of the wet film formation method include inkjet methods, spray methods, nozzle print methods, spin coating methods, dip coating methods, casting methods, die coating methods, roll coating methods, bar coating methods and gravure coating methods.

The photoelectric conversion element of the present embodiment may have single or two or more auxiliary layers other than the first auxiliary layer 103 between the first auxiliary layer 103 and the lower electrode 102. Examples of such an auxiliary layer include hole injection layers that improve hole injectability from the lower electrode 102 to the first auxiliary layer 103. Examples of the material constituting the hole injection layer include phthalocyanine derivatives, starburst amines such as m-MTDATA (4,4′,4″-tris[phenyl(m-tolyl)amino]triphenylamine), and polymer materials including polythiophene and polyvinylcarbazole derivatives, such as PEDOT (poly(3,4-ethylenedioxythiophene)). The thickness of this auxiliary layer may be the same as that of the first auxiliary layer 103.

The photoelectric conversion element of the present embodiment may have single or two or more auxiliary layers other than the second auxiliary layer 105 between the second auxiliary layer 105 and the upper electrode 106. Examples of such an auxiliary layer include electron injection layers that improve electron injectability from the upper electrode 106 to the second auxiliary layer 105, and electron transport layers. Examples of the material constituting the electron injection layer include metals such as cesium, lithium and strontium, and lithium fluoride. The material constituting the electron transport layer may be the same as described above. The thickness of this auxiliary layer may be the same as that of the second auxiliary layer 105.

The photoelectric conversion element of the present embodiment may have, in addition to each of the layers mentioned above, at least one of an interlayer contact improvement layer and a crystallization prevention layer positioned between the layers.

The interlayer contact improvement layer has a function of reducing damage on a lower film nearest to an upper electrode, for example, the photoelectric conversion film 110, at the time of film formation of the upper electrode 106. Particularly, high-energy particles present in an apparatus for use in the film formation of the upper electrode 106 to be formed, for example, sputter particles, secondary electrons, Ar particles, or oxygen anions in a sputtering method, may deteriorate the lower film nearest thereto through collision, resulting in performance deterioration such as increased leak current or reduced sensitivity. One of the methods for preventing this preferably involves establishing an interlayer contact improvement layer on an upper layer of the nearest lower film. An organic material such as copper phthalocyanine, NTCDA, PTCDA, [dipyrazino[2,3-F:2′,3′-H]quinoxaline-2,3,6,7,10,11-hexacarbonitrile](HATCN), an acetyl acetonate complex, or BCP, an organic metal compound, or an inorganic material such as MgAg or MgO is preferably used as the material for the interlayer contact improvement layer. The thickness of the interlayer contact improvement layer differs in proper range depending on the configuration of a photoelectric conversion film, the film thickness of an electrode, etc. It is particularly preferred to select a material having no absorption in the visible region, or the thickness is preferably 2 nm or more and 500 nm or less from the viewpoint of using a small thickness.

As mentioned above, the photoelectric conversion element of the present embodiment is connected with an accumulation unit serving as a condenser for accumulation of the generated charge and a readout unit serving as a transistor circuit for readout via a connection unit made of a conductive material. Also, the photoelectric conversion element optionally contains a protective structure against outside air, such as a protective film, a substrate for strength retention, a microlens for light collection, or the like.

The readout unit is disposed in order to read out signals depending on a charge generated in the photoelectric conversion film. The readout unit is constituted by, for example, CCD, a CMOS circuit, or a TFT circuit, and preferably shielded from lights by a light shielding layer disposed in an insulating layer. The readout circuit is electrically connected with its corresponding electrode via a connection unit. In order to secure a charge in a quantity necessary for readout, an accumulation unit constituted by a condenser or the like may intervene between the electrode and the connection unit. The connection unit is embedded in the insulating layer and is a plug or the like for electrically connecting the electrode (e.g., a transparent electrode or an opposite electrode) to the readout unit. When the member thus configured is a solid image sensor, upon light incidence, the light is incident on the photoelectric conversion film where a charge is then generated. Electrons in the generated charge are collected (and accumulated) in one electrode, and holes are collected in the other electrode. Voltage signals depending on the quantity thereof is output to the outside of the solid image sensor by the readout unit.

### (Image Sensor)

The image sensor of the present embodiment may have the same configuration as that of a conventional image sensor except that the image sensor has the photoelectric conversion element of the present embodiment. The image sensor of the present embodiment has, for example, a large number of photoelectric conversion elements of the present embodiment disposed in an array pattern. Specifically, a large number of photoelectric conversion elements disposed in an array pattern constitute a solid image sensor that exhibits the quantity of an incident light as well as positional information on incidence.

The image sensor of the present embodiment may have one photoelectric conversion element of the present embodiment or may be prepared by laminating two or more photoelectric conversion elements of the present embodiment. In the case of laminating two or more photoelectric conversion elements of the present embodiment, the respective photoelectric conversion elements may perform photoelectric conversion by selectively detecting lights at wavelength bands different from each other. In the case of laminating three or more photoelectric conversion elements of the present embodiment, at least one thereof obtains green color signals, at least one of the remaining photoelectric conversion elements obtains blue color signals, at least one of the remaining photoelectric conversion elements obtains red color signals, and at least one of the remaining photoelectric conversion elements obtains color signals of infrared light. The image sensor can thereby obtain plural types of color signals by one pixel without the use of a color filter. Color signals other than those to be detected by the photoelectric conversion elements of the present embodiment may be sensed by a device having a heretofore known silicon photodiode.

In the image sensor, a plurality of photoelectric conversion elements and a device having a silicon photodiode may be laminated as long as a photoelectric conversion element disposed nearer a light source does not block (i.e., transmissive to) the absorption wavelength of another photoelectric conversion element disposed at the back thereof viewed from the light source side.

In the image sensor, the photoelectric conversion elements may be partially constituted as a thin film in the same plane having no structural separation between the adjacent photoelectric conversion elements, from the viewpoint of easy molding.

The image sensor of the present embodiment may further include a substrate. The substrate is used for producing the image sensor by laminating each layer thereon, or used for enhancing the mechanical strength of the image sensor. Examples of the type of the substrate include, but are not particularly limited to, semiconductor substrates, glass substrates and plastic substrates.

### (Photosensor)

The photosensor of the present embodiment may have the same configuration as that of a conventional photosensor except that the photosensor has the image sensor of the present embodiment. This photosensor receives a light by the image sensor of the present embodiment and can output electric signals in response to the amount of the light received.

### (Solid-State Imaging Apparatus)

The solid-state imaging apparatus of the present embodiment may have the same configuration as that of a conventional solid-state imaging apparatus except that the solid-state imaging apparatus has the image sensor of the present embodiment. The solid-state imaging apparatus of the present embodiment may be, for example, a CMOS image sensor, and may have a pixel unit as an imaging area on a semiconductor substrate and further have a peripheral circuit unit having a row scanning unit, a horizontal selection unit, a column scanning unit, and a system control unit, in a peripheral region of or beneath in a vertical direction this pixel unit. The pixel unit has the image sensor of the present embodiment.

The photoelectric conversion element of the present embodiment employs the material for a photoelectric conversion element of the present embodiment and thereby has the following advantages: the photoelectric conversion element of the present embodiment has a low dark current value because a short circuit or a pinhole is unlikely to occur. As a result, the photoelectric conversion element of the present embodiment has excellent leak-preventing properties (particularly, in the dark). Moreover, the photoelectric conversion element of the present embodiment tends to easily exhibit a high light/dark ratio and in this case, has better leak-preventing properties. Also, the photoelectric conversion element of the present embodiment is excellent in hole- and electron-transporting properties, despite the material for a photoelectric conversion element that is unlikely to aggregate, and therefore has high photoelectric conversion efficiency. Besides, the photoelectric conversion element of the present embodiment employs the material for a photoelectric conversion element of the present embodiment and thereby has favorable heat resistance, and its durability in a production process and an environment of actual use is improved.

### EXAMPLES

Hereinafter, the present invention will be described in more detail with reference to Examples. However, the present invention is not limited by these Examples. Synthesized compounds were further purified by sublimation, if necessary.

### Synthesis Example 1

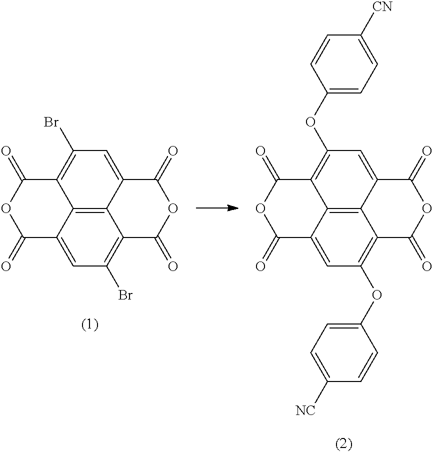 [SMILES: O=C1OC(=O)c2c(Br)cc3c4c(c(Br)cc1c24)C(=O)OC3=O ; N#Cc1ccc(Oc2cc3c4c(c(Oc5ccc(C#N)cc5)cc5c4c2C(=O)OC5=O)C(=O)OC3=O)cc1]

A mixture of 1.5 g of 2,6-dibromonaphthalene-1,4,5,8-tetracarboxylic dianhydride (manufactured by Tokyo Chemical Industry Co., Ltd.), 1.0 g of 4-cyanophenol (manufactured by Tokyo Chemical Industry Co., Ltd.), and 7 mL of isoquinoline (manufactured by Tokyo Chemical Industry Co., Ltd.) was stirred at 180° C. for 5 hours. Subsequently, the mixture was cooled to room temperature, and methanol was then added thereto. A deposited mixture was filtered. The mixture was further washed with methanol to obtain a compound (2).

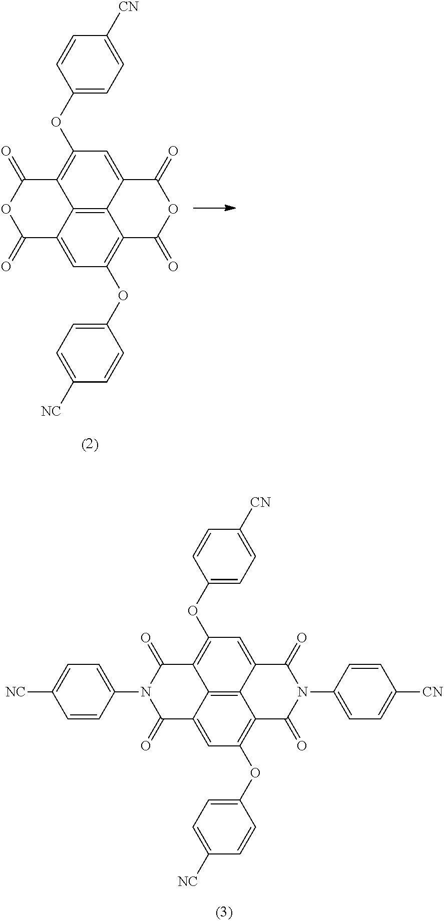 [SMILES: N#Cc1ccc(Oc2cc3c4c(c(Oc5ccc(C#N)cc5)cc5c4c2C(=O)OC5=O)C(=O)OC3=O)cc1 ; N#Cc1ccc(Oc2cc3c4c(c(Oc5ccc(C#N)cc5)cc5c4c2C(=O)N(c2ccc(C#N)cc2)C5=O)C(=O)N(c2ccc(C#N)cc2)C3=O)cc1]

A mixture obtained by adding 1.0 g of the compound (2) and 0.5 g (2.1 molar equivalent based on the compound (2)) of 4-aminobenzonitrile (manufactured by Tokyo Chemical Industry Co., Ltd.) to 10 mL of acetic acid (manufactured by Tokyo Chemical Industry Co., Ltd.) was stirred at 125° C. for 8 hours. Then, the mixture was cooled to room temperature, and methanol was added thereto. A deposited mixture was filtered. The mixture was further washed with methanol to obtain a compound (3). Results of NMR measurement thereof are shown below.

¹HNMR (500 MHz, DMSO-d6): 8.35 (s, 2H), 8.02 (d, 4H), 7.91 (d, 4H), 7.63 (d, 4H), 7.21 (d, 4H)

### Synthesis Example 2

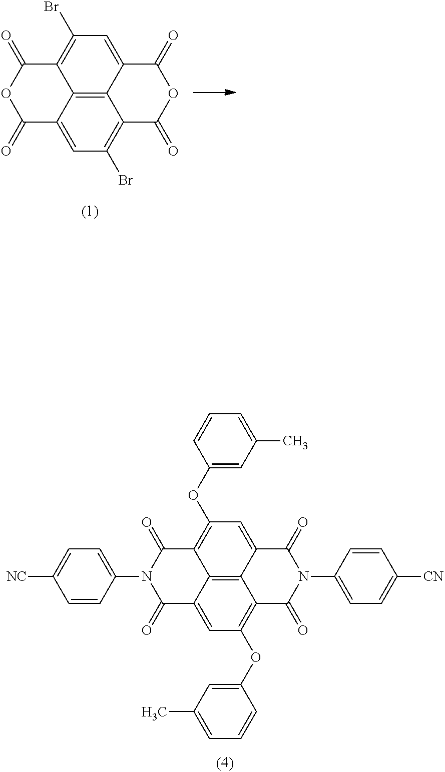 [SMILES: O=C1OC(=O)c2c(Br)cc3c4c(c(Br)cc1c24)C(=O)OC3=O ; Cc1cccc(Oc2cc3c4c(c(Oc5cccc(C)c5)cc5c4c2C(=O)N(c2ccc(C#N)cc2)C5=O)C(=O)N(c2ccc(C#N)cc2)C3=O)c1]

A compound (4) was obtained in the same manner as in Synthesis Example 1 except that m-cresol (manufactured by FUJIFILM Wako Pure Chemical Corp.) was used instead of 4-cyanophenol. Results of NMR measurement thereof are shown below.

¹HNMR (500 MHz, DMSO-d6): 8.06 (s, 2H), 8.02 (d, 4H), 7.65 (d, 4H), 7.37 (m, 1H), 7.11 (d, 1H), 7.00 (s, 1H), 6.96 (dd, 1H), 2.32 (s, 6H)

### Synthesis Example 3

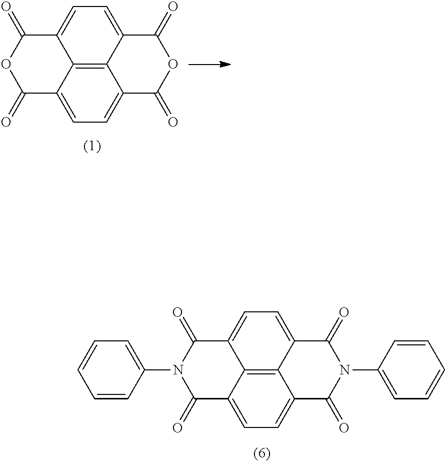 [SMILES: O=C1OC(=O)c2ccc3c4c(ccc1c24)C(=O)OC3=O ; O=C1c2ccc3c4c(ccc(c24)C(=O)N1c1ccccc1)C(=O)N(c1ccccc1)C3=O]

A mixture obtained by adding 2.0 g of 1,4,5,8-naphthalenetetracarboxylic dianhydride (hereinafter, referred to as a “compound (5)” (manufactured by Tokyo Chemical Industry Co., Ltd.)) and 1.5 g (2.2 molar equivalent based on the compound (5)) of aniline (manufactured by Tokyo Chemical Industry Co., Ltd.) to 15 mL of acetic acid (manufactured by Tokyo Chemical Industry Co., Ltd.) was stirred under reflux at 125° C. for 8 hours. Then, the mixture was cooled to room temperature, and methanol was added thereto. A deposited mixture was filtered. The mixture was further washed with methanol. Then, pyridine was added thereto, and the mixture was stirred for 5 minutes. Subsequently, the mixture was filtered, washed with methanol, and subjected to sublimation and purification to obtain a white solid compound (6). Results of NMR measurement thereof are shown below.

¹HNMR (500 MHz, DMSO-d6): 8.73 (s, 4H), 7.58-7.45 (m, 10H)

### Synthesis Example 4

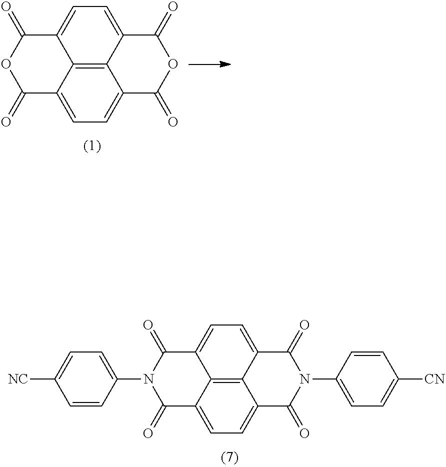 [SMILES: O=C1OC(=O)c2ccc3c4c(ccc1c24)C(=O)OC3=O ; N#Cc1ccc(N2C(=O)c3ccc4c5c(ccc(c35)C2=O)C(=O)N(c2ccc(C#N)cc2)C4=O)cc1]

A mixture obtained by adding 6.0 g of the compound (5), 6.1 g (2.1 molar equivalent based on the compound (5)) of isoquinoline (manufactured by Tokyo Chemical Industry Co., Ltd.), and 6.6 g (2.5 molar equivalent based on the compound (5)) of 4-aminobenzonitrile (manufactured by Tokyo Chemical Industry Co., Ltd.) to 90 mL of m-cresol (manufactured by Tokyo Chemical Industry Co., Ltd.) was stirred at 180° C. for 8 hours. Then, the mixture was cooled to room temperature, and methanol was added thereto. A deposited mixture was filtered. After further washing with methanol, an aqueous potassium carbonate solution was added thereto, and the mixture was stirred for 5 minutes. Subsequently, the mixture was filtered, then washed with water, methanol, and purified by sublimation to obtain a white solid compound (7). Results of NMR measurement thereof are shown below.

¹HNMR (500 MHz, HFIP-d2): 8.93 (s, 4H), 8.77 (dm, 4H), 7.55 (dm, 4H)

### [Crystallinity Evaluation of Compound]

The synthesized compounds were evaluated for their crystallinity. In the crystallinity evaluation, a desktop X-ray diffraction apparatus (manufactured by Rigaku Holdings Corp., product name (MiniFlex600)) was used. The X-ray source used was CuKα, and the maximum peak intensity of X-ray diffraction (XRD) was measured from a spectrum obtained at a scan rate of 20°/min. The results are shown in Table 1. The maximum peak intensity is indicated in a relative value with the value of the compound (4) defined as 1.

### [Preparation and Evaluation of Organic Thin Film and Photoelectric Conversion Element]

In Examples and Comparative Examples given below, an organic thin film and a photoelectric conversion element were prepared using a vapor deposition machine, and the application and measurement of current and voltage were performed in air. The prepared photoelectric conversion element was placed in a measurement chamber, and the application and measurement of current and voltage were performed. The application and measurement of current and voltage employed a semiconductor parameter analyzer (manufactured by Keithley Instruments). Irradiation with incident light was performed under conditions involving an irradiation light wavelength of 550 nm and an irradiation light half-value width of 20 nm using a light source apparatus (manufactured by Asahi Spectra Co., Ltd., product name (PVL-3300)). A light-dark ratio is a value determined by dividing a current value in the case of light irradiation by a current value in the dark.

### Example 1

On ITO transparent conductive glass (ITO manufactured by GEOMATEC Co., Ltd., thickness: 100 nm), a film of boron subphthalocyanine chloride (purified product manufactured by Sigma-Aldrich Co., LLC., purity >99%, LUMO: −2.6 eV, difference in energy level: 2.7 eV) was formed in vacuum as a photoelectric conversion layer having a thickness of 100 nm. A film of a sublimation-purified product of tris(8-quinolinolato)aluminum (Alq₃) (manufactured by Tokyo Chemical Industry Co., Ltd.) was formed thereon as an auxiliary layer 1 having a thickness of 25 nm by resistance heating vacuum vapor deposition. Subsequently, a film of the compound (3) was formed thereon as an auxiliary layer 2 having a thickness of 25 nm by resistance heating vacuum vapor deposition. Subsequently, on the auxiliary layer 2, a film of aluminum was formed in vacuum as an electrode having a thickness of 100 nm to prepare a photoelectric conversion element. A voltage of 5 V was applied to the obtained photoelectric conversion element using the ITO and aluminum as electrodes, and a current value in the dark and a current value at the time of light irradiation were measured. A light/dark ratio was calculated from the measurement results. The results are shown in Table 1. The dark current value is indicated by a relative value to a value defined as 1 in Comparative Example 1 mentioned later.

### Example 2

A single-layer organic thin film and a photoelectric conversion element were prepared in the same manner as in Example 1 except that the compound (4) was used instead of the compound (3). The obtained photoelectric conversion element was evaluated in the same manner as in Example 1. The results are shown in Table 1.

### Comparative Example 1

A single-layer organic thin film and a photoelectric conversion element were prepared in the same manner as in Example 1 except that the compound (5) was used instead of the compound (3). The obtained photoelectric conversion element was evaluated in the same manner as in Example 1. The results are shown in Table 1.

### Comparative Example 2

A single-layer organic thin film and a photoelectric conversion element were prepared in the same manner as in Example 1 except that the compound (6) was used instead of the compound (3). The obtained photoelectric conversion element was evaluated in the same manner as in Example 1. The results are shown in Table 1.

### Comparative Example 3

A single-layer organic thin film and a photoelectric conversion element were prepared in the same manner as in Example 1 except that the compound (7) was used instead of the compound (3). The obtained photoelectric conversion element was evaluated in the same manner as in Example 1. The results are shown in Table 1.

TABLE 1

| | | | | | Dark current | |
|---|---|---|---|---|---|---|
| | | | Difference in | Maximum peak | value (relative | Light/dark ratio |
| | | LUMO(eV) | energy level | intensity of XRD | value) | (relative value) |
| | | DFT | (ev) | (relative value) | At time of | At time of |
| | | calculated | DFT calculated | XRD | application of 5 | application of 5 |
| | Compound | value | value | measurement | V | V |
| Example 1 | (3) | −3.90 | 3.10 | 0.1 | 0.0001 | 3393 |
| Example 2 | (4) | −3.40 | 3.15 | 0.4 | 0.002 | 357 |
| Comparative | (5) | −4.00 | 3.70 | 1 | 1 | 1 |
| Example 1 | | | | | | |
| Comparative | (6) | −3.35 | 3.60 | 0.5 | 17 | 0.1 |
| Example 2 | | | | | | |
| Comparative | (7) | −3.82 | 3.54 | 0.1 | 218 | 0.08 |
| Example 3 | | | | | | |

The results shown in Table 1 revealed that the photoelectric conversion element of the present invention exhibits a low dark current value and therefore has excellent leak-preventing properties (particularly, in the dark). Particularly, in Example 1, the maximum peak intensity of XRD was small, leading to low crystallinity and low LUMO. Also, the difference in energy level was large, and a high light/dark ratio was therefore exhibited, demonstrating that the compound had much better leak-preventing properties. The compound of the present invention was found to be suitable as a material for a photoelectric conversion element, particularly, a material contained in an electron transport layer and a hole blocking layer of a photoelectric conversion element.

### INDUSTRIAL APPLICABILITY

Use of a material for a photoelectric conversion element containing the compound (1) of the present invention can provide a photoelectric conversion element excellent in required performance such as leak-preventing properties and transporting properties of holes and electrons, heat resistance, and visible light transparency. Thus, the compound (1), the material for a photoelectric conversion element, the organic thin film and the photoelectric conversion element of the present invention have industrial applicability in fields that require these characteristics. Specifically, the compound (1) and the photoelectric conversion element of the present invention have industrial applicability as a solid image sensor in, for example, image sensors for security cameras, in-car cameras, cameras for uninhabited air vehicles, agricultural cameras, industrial cameras, medical cameras such as endoscopic cameras, cameras for game machines, digital still cameras, digital video cameras, cameras for mobile phones, and cameras for the other mobile instruments; image scanning elements for facsimiles, scanners and copying machines; and photosensors for biosensors and chemical sensors. Also, the compound (1), the material for a photoelectric conversion element, the organic thin film and the photoelectric conversion element of the present invention have industrial applicability as a display using electroluminescence in, for example, television monitors, touch monitors, digital signages, wearable displays, electronic papers, and head-up displays for mobility application.

The present application is based on Japanese Patent Application No. 2023-069028 filed on Apr. 20, 2023, the contents of which are incorporated herein by reference.

### REFERENCE SIGNS LIST

100 . . . photoelectric conversion element, 101 . . . base material, 102 . . . lower electrode, 103 . . . first auxiliary layer, 104 . . . photoelectric conversion layer, 105 . . . second auxiliary layer, 106 . . . upper electrode, 110 . . . photoelectric conversion film.

1. An organic thin film comprising a compound represented by the following formula (1): 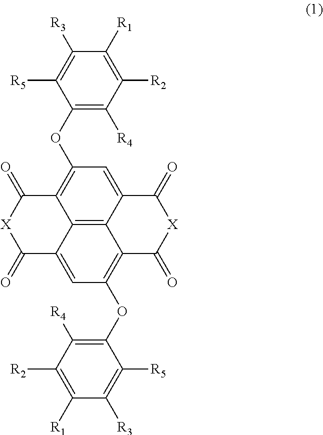
wherein
X is an oxygen atom or NR₆,
R₁, R₂, R₃, R₄, R₅ and R₆ are each independently selected from the group consisting of a hydrogen atom, a halogen atom, a hydroxy group, a thiol group, an amino group, a cyano group, a carboxy group, a nitro group, and an optionally substituted linear, branched or cyclic alkyl group, thioalkyl group, thioaryl group, arylsulfonyl group, aryloxy group, alkylsulfonyl group, alkylamino group, arylamino group, alkoxy group, acylamino group, acyloxy group, aryl group, carboxyamide group, carboalkoxy group, carboaryloxy group, acyl group, and monovalent heterocyclic group,
any adjacent members among R₁, R₂, R₃, R₄, R₅ and R₆ optionally constitute a portion of a condensed aliphatic ring or a condensed aromatic ring, and
two R₁, R₂, R₃, R₄, R₅ or X moieties are the same as or different from each other, wherein the condensed aliphatic ring and the condensed aromatic ring each optionally contain one or more atoms other than carbon.

2. The organic thin film according to claim 1, wherein an energy level of the lowest unoccupied molecular orbital obtained by density functional formalism of the compound represented by the formula (1) is −6.00 eV or more and −3.40 eV or less.

3. The organic thin film according to claim 1, wherein a difference between an energy level of the lowest unoccupied molecular orbital and an energy level of the highest occupied molecular orbital obtained by density functional formalism of the compound represented by the formula (1) is 3.00 eV or more and 4.00 eV or less.

4. The organic thin film according to claim 1, wherein the compound represented by the formula (1) is a material for a photoelectric conversion element.

5. (canceled)

6. The organic thin film according to claim 1, wherein the organic thin film has a local maximum absorption wavelength of an optical absorption band at 450 nm or less.

7. (canceled)

8. A photoelectric conversion element comprising a first electrode film, a second electrode film, and a photoelectric conversion film positioned between the first electrode film and the second electrode film,
wherein the photoelectric conversion film comprises the organic thin film according to claim 1.

9. The photoelectric conversion element according to claim 8, wherein
the photoelectric conversion film comprises a photoelectric conversion layer and an auxiliary layer, wherein
the auxiliary layer is made of only the organic thin film or made of a plurality of films including the organic thin film.

10. An image sensor comprising the photoelectric conversion element according to claim 8.

11. The image sensor according to claim 10, wherein the image sensor is prepared by laminating two or more photoelectric conversion elements.

12. An image sensor prepared by disposing a plurality of photoelectric conversion elements according to claim 8 in an array pattern.

13. A photosensor comprising the image sensor according to claim 10.

14. A solid-state imaging apparatus comprising the image sensor according to claim 10.

15. An image sensor comprising the photoelectric conversion element according to claim 9.

16. The image sensor according to claim 15, wherein the image sensor is prepared by laminating two or more photoelectric conversion elements.

17. An image sensor prepared by disposing a plurality of photoelectric conversion elements according to claim 9 in an array pattern.

18. A photosensor comprising the image sensor according to claim 15.

19. A solid-state imaging apparatus comprising the image sensor according to claim 15.
